(19) United States
(12) Patent Application Publication
(10) Pub. No.: US 2019/0203295 A1
(43) Pub. Date: Jul. 4, 2019

(54) METHODS OF PREDICTING COMPLICATION AND SURGERY IN CROHN'S DISEASE

(71) Applicant: CEDARS-SINAI MEDICAL CENTER, Los Angeles, CA (US)

(72) Inventors: Jerome I. Rotter, Los Angeles, CA (US); Kent D. Taylor, Ventura, CA (US); Marla Dubinsky, Los Angeles, CA (US); Stephan R. Targan, Santa Monica, CA (US)

(21) Appl. No.: 16/355,376

(22) Filed: Mar. 15, 2019

Related U.S. Application Data
(63) Continuation of application No. 13/263,707, filed on Dec. 15, 2011, now abandoned.
(60) Provisional application No. 61/167,752, filed on Apr. 8, 2009.

(51) Int. Cl. C12Q 1/6883

(52) U.S. Cl. CPC C12Q 1/6883; C12Q 2600/118; C12Q 2600/106; C12Q 2600/156

(57) ABSTRACT

The present invention relates to prognosing, diagnosing and treating an aggressive form of Crohn's disease characterized by rapid progression to complication and/or surgery from the time of diagnosis. In one embodiment, the prognosis, diagnosis and treatment is based upon the presence of one or more genetic risk factors.

### FIELD OF THE INVENTION

The invention relates generally to the field of inflammatory disease, specifically to Crohn's disease and progression to complication and/or surgery.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

Thus, there is a need in the art to identify environmental factors, serological profiles, genes, allelic variants and/or haplotypes that may assist in explaining the genetic risk, diagnosing and/or predicting susceptibility for or protection against inflammatory bowel disease.

### BRIEF DESCRIPTION OF THE FIGURES

FIG. 1 depicts, in accordance with an embodiment herein, survival distribution for subgroups of SC1 (model 1) for survival for complication.

FIG. 2 depicts, in accordance with an embodiment herein, survival distribution for subgroups of SC2 (model 2) for survival for complication.

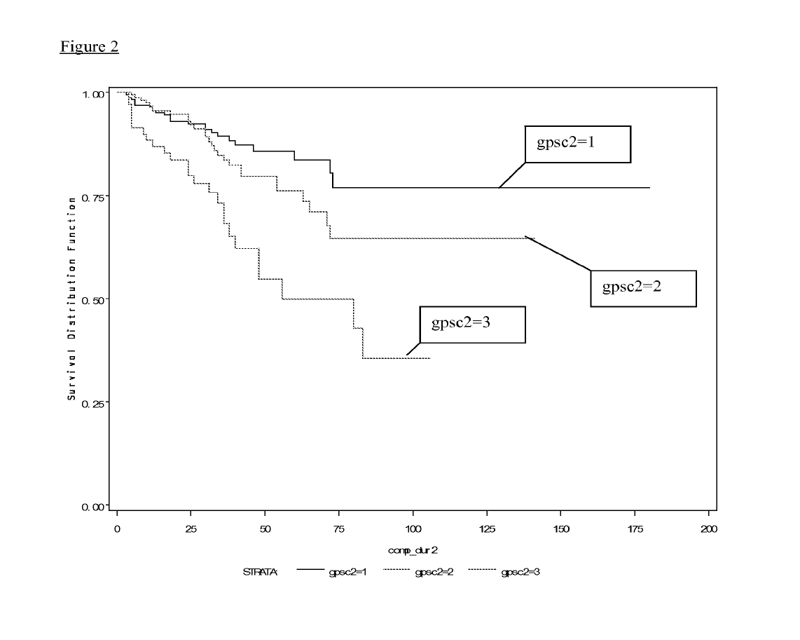

FIG. 3 depicts, in accordance with an embodiment herein, survival distribution across models for stratum 1 for survival for complication.

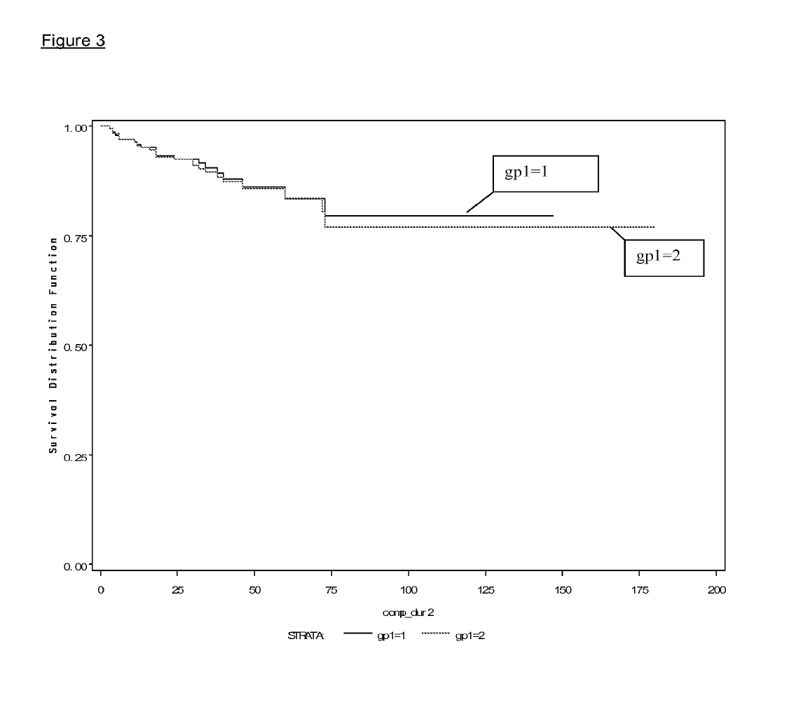

FIG. 4 depicts, in accordance with an embodiment herein, survival distribution across models for stratum 2 for survival for complication.

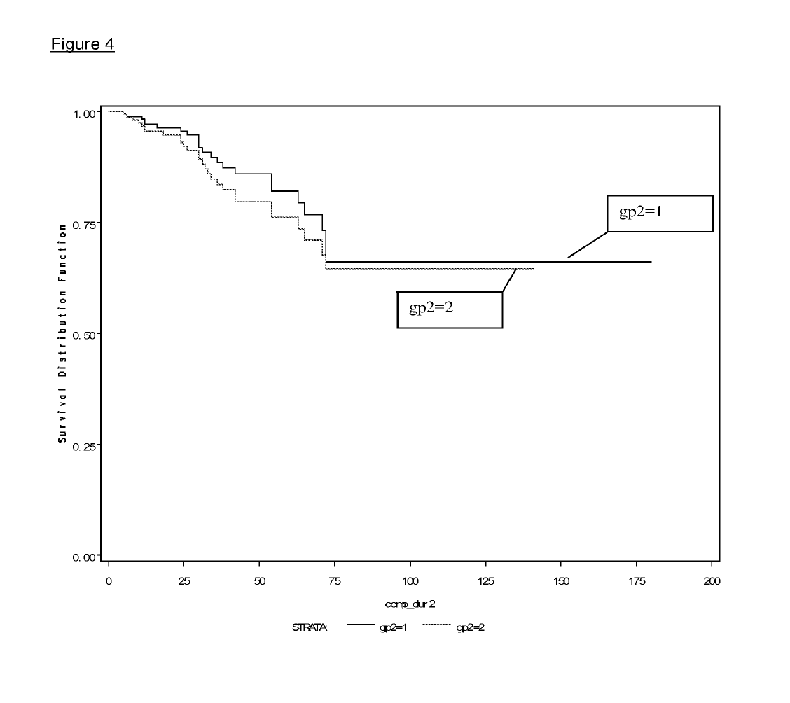

FIG. 5 depicts, in accordance with an embodiment herein, survival distribution across models for stratum 3 for survival for complication.

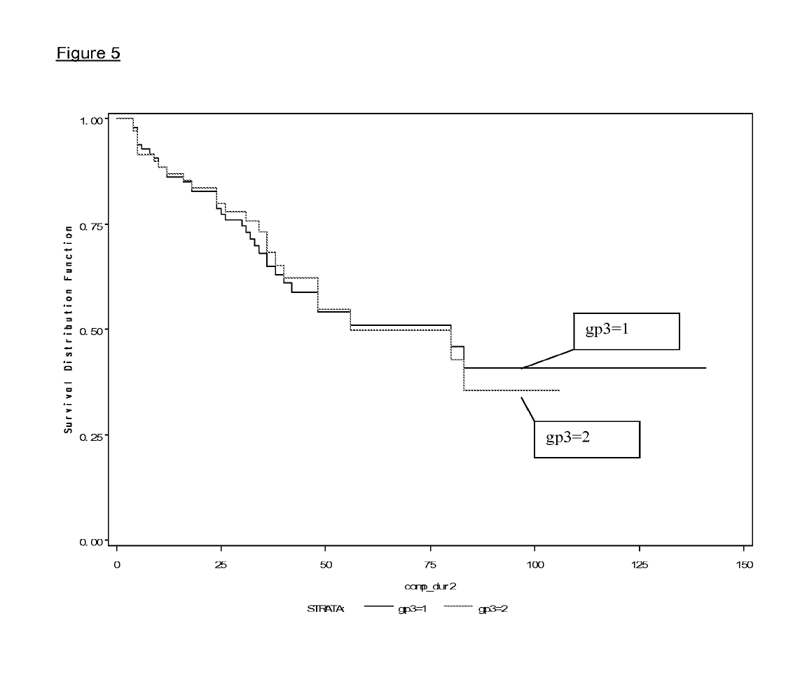

FIG. 6 depicts, in accordance with an embodiment herein, survival distribution for subgroups of SS1 (model 1) for survival for surgery.

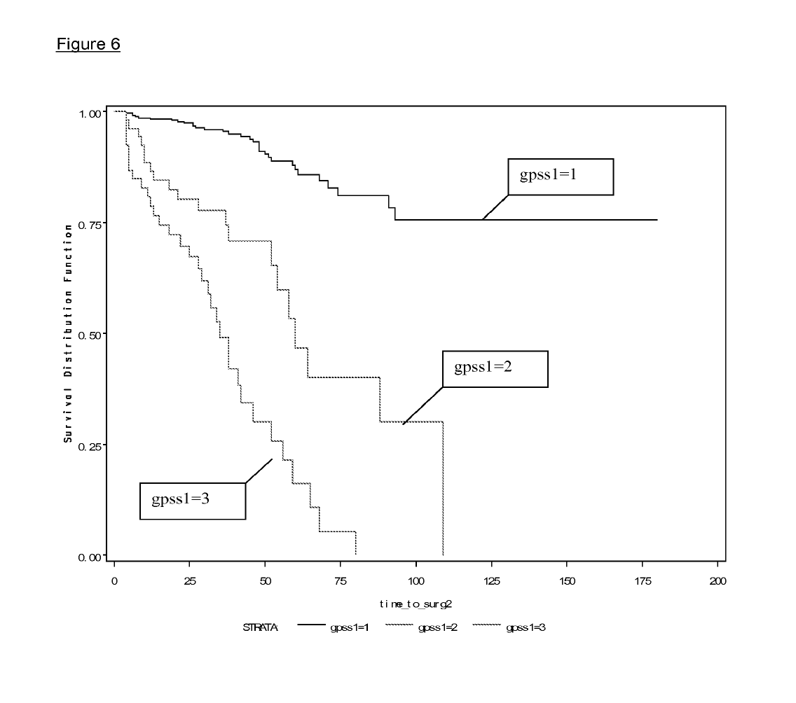

FIG. 7 depicts, in accordance with an embodiment herein, survival distribution for subgroups of SS2 (model 2) for survival for surgery.

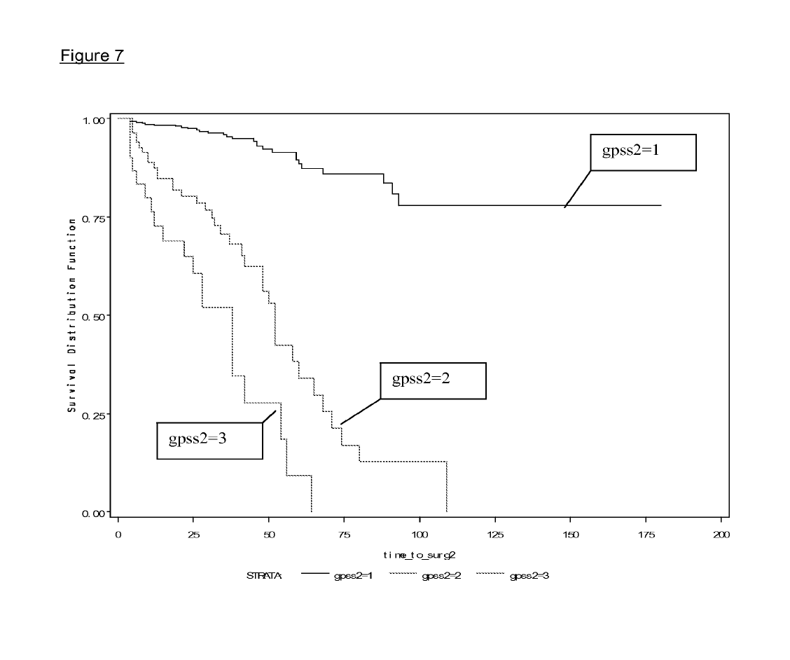

FIG. 8 depicts, in accordance with an embodiment herein, survival distribution for subgroups of SS3 (model 3) for survival for surgery.

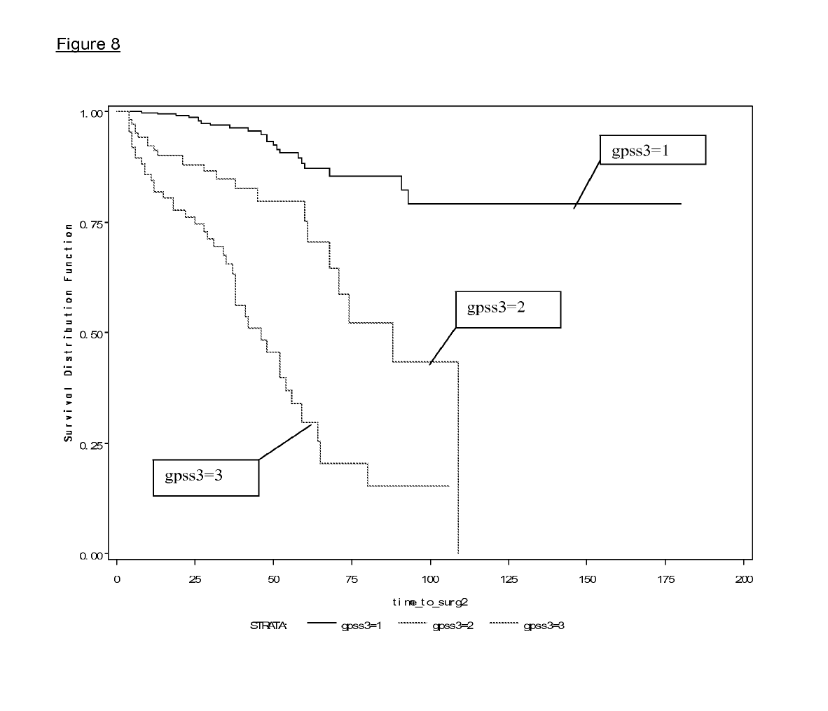

FIG. 9 depicts, in accordance with an embodiment herein, survival distribution for subgroups of SS4 (model 4) for survival for surgery.

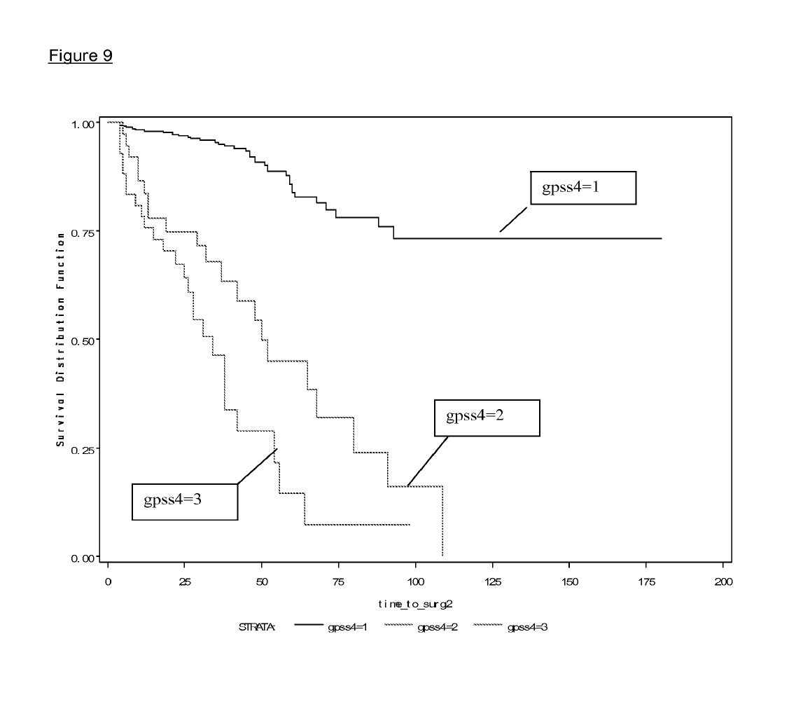

FIG. 10 depicts, in accordance with an embodiment herein, survival distribution across models for stratum 1 for survival for surgery.

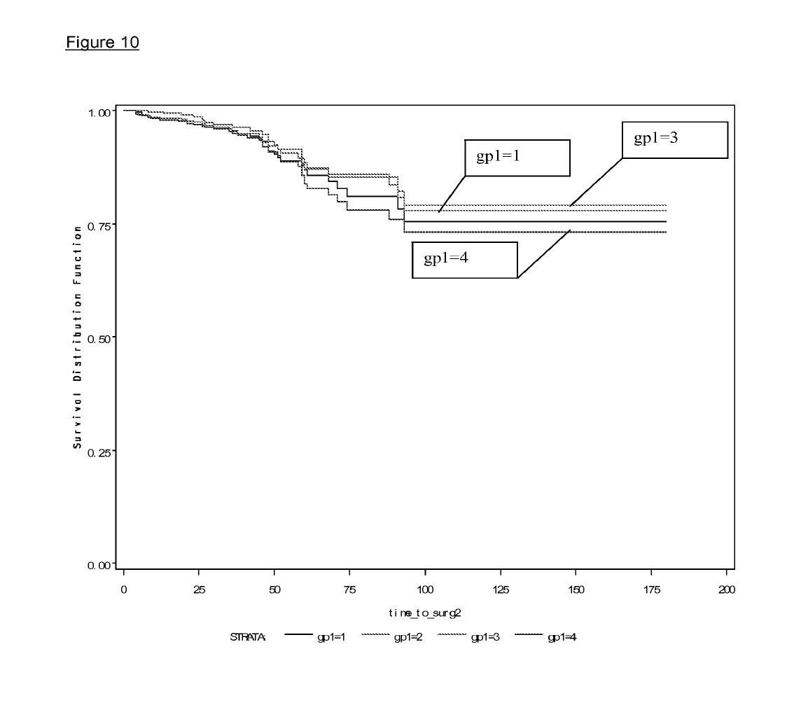

FIG. 11 depicts, in accordance with an embodiment herein, survival distribution across models for stratum 2 for survival for surgery.

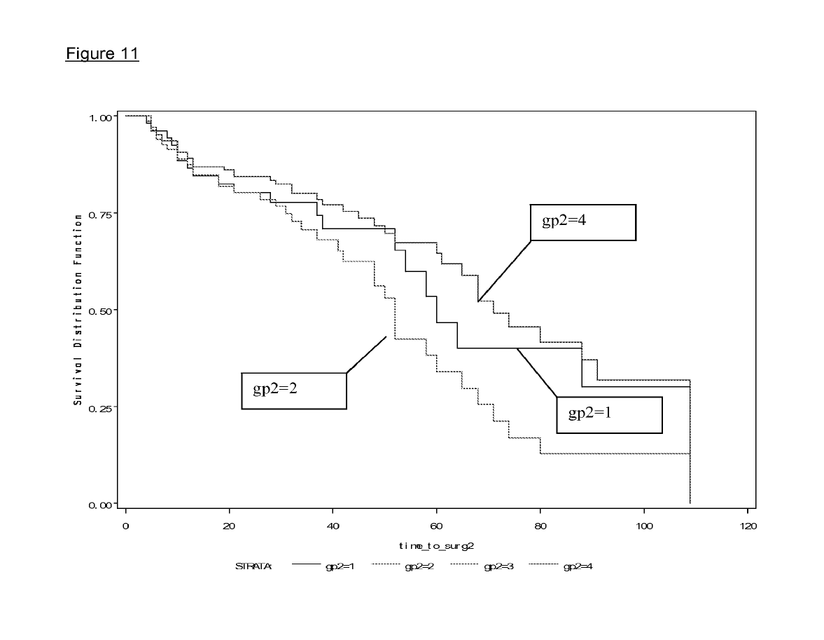

FIG. 12 depicts, in accordance with an embodiment herein, survival distribution across models for stratum 3 for survival for surgery.

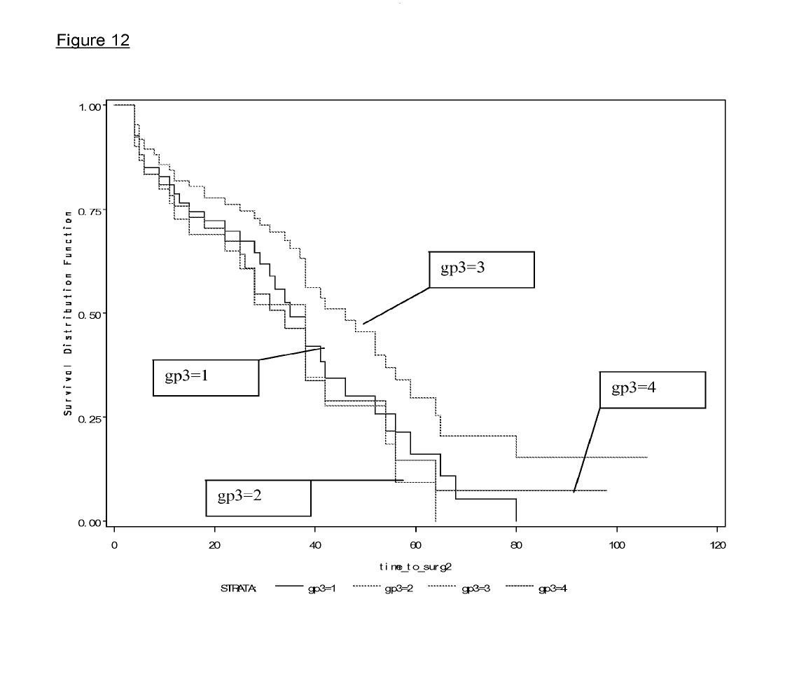

### SUMMARY OF THE INVENTION

Various embodiments include a method of prognosing Crohn's disease in an individual, comprising obtaining a sample from the individual, assaying the sample for the presence or absence of one or more genetic risk variants, and prognosing an aggressive form of Crohn's disease based on the presence of one or more genetic risk variants, where the one or more genetic risk variants are selected from the genetic loci of 8q24, 16p11, Bromodomain and WD repeat domain containing 1 (BRWD1) and/or Tumor necrosis factor superfamily member 15 (TNFSF15). In another embodiment, the presence of each genetic risk variant has an additive effect on rapidity of Crohn's disease progression from a relatively less severe case of Crohn's disease to a relatively more severe case of Crohn's disease. In another embodiment, the one or more genetic risk variants comprise SEQ. ID. NO.: 1, SEQ. ID. NO.: 2, SEQ. ID. NO.: 3, SEQ. ID. NO.: 4, SEQ. ID. NO.: 5 and/or SEQ. ID. NO.: 6. In another embodiment, the aggressive form of Crohn's disease is characterized by one or more phenotypes associated with complications. In another embodiment, the aggressive form of Crohn's disease is characterized by one or more phenotypes associated with conditions requiring surgery. In another embodiment, the aggressive form of Crohn's Disease is characterized by a rapid progression from a relatively less severe case of Crohn's disease to a relatively more severe case of Crohn's disease. In another embodiment, the individual has previously been diagnosed with inflammatory bowel disease (IBD). In another embodiment, the individual is a child 17 years old or younger. In another embodiment, the aggressive form of Crohn's disease comprises internal penetrating and/or stricture. In another embodiment, the aggressive form of Crohn's disease comprises a high expression of anti-neutrophil cytoplasmic antibody (ANCA) relative to levels found in a healthy individual. In another embodiment, the presence of one or more genetic risk variants is determined from an expression product thereof.

Other embodiment include a method of prognosing Crohn's disease in an individual, comprising obtaining a sample from the individual, assaying the sample for the presence or absence of one or more genetic risk variants, and prognosing a form of Crohn's disease associated with a complication based on the presence of one or more genetic risk variants, where the one or more genetic risk variants is selected from the group consisting of SEQ. ID. NO.: 7, SEQ. ID. NO.: 8, SEQ. ID. NO.: 9, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, SEQ. ID. NO.: 18, SEQ. ID. NO.: 19, SEQ. ID. NO.: 20, SEQ. ID. NO.: 21, and/or SEQ. ID. NO.: 22. In another embodiment, the complication comprises internal penetrating and/or stricturing disease.

Other embodiments include a method of prognosing Crohn's disease in an individual, comprising obtaining a sample from the individual, assaying the sample for the presence or absence of one or more genetic risk variants, and prognosing a form of Crohn's disease associated with one or more conditions that require a treatment by surgery, where the one or more genetic risk variants is selected from the group consisting of SEQ. ID. NO.: 23, SEQ. ID. NO.: 24, SEQ. ID. NO.: 25, SEQ. ID. NO.: 26, SEQ. ID. NO.: 27, SEQ. ID. NO.: 28, SEQ. ID. NO.: 29, SEQ. ID. NO.: 30, SEQ. ID. NO.: 31, SEQ. ID. NO.: 32, SEQ. ID. NO.: 33, SEQ. ID. NO.: 34, SEQ. ID. NO.: 35, SEQ. ID. NO.: 36, SEQ. ID. NO.: 37, SEQ. ID. NO.: 38, SEQ. ID. NO.: 39, SEQ. ID. NO.: 40, SEQ. ID. NO.: 41, SEQ. ID. NO.: 42, SEQ. ID. NO.: 43, SEQ. ID. NO.: 44, SEQ. ID. NO.: 45, SEQ. ID. NO.: 46, SEQ. ID. NO.: 47, SEQ. ID. NO.: 48, SEQ. ID. NO.: 49, SEQ. ID. NO.: 50, SEQ. ID. NO.: 51, and/or SEQ. ID. NO.: 52. In another embodiment, the treatment by surgery comprises small-bowel resection, colectomy and/or colonic resection.

Various embodiments include a method of treating Crohn's disease in an individual, comprising prognosing an aggressive form of Crohn's disease in the individual based on the presence of one or more genetic risk variants, and treating the individual, where the one or more genetic risk variants are selected from the genetic loci of 8q24, 16p11, Bromodomain and WD repeat domain containing 1 (BRWD1) and/or Tumor necrosis factor superfamily member 15 (TNFSF15). In another embodiment, treating the individual comprises exposing the individual to a treatment that ameliorates the symptoms of Crohn's disease on the basis that the subject tests positive for one or more genetic risk variants. In another embodiment, treating the individual comprises administering a surgical procedure associated with treating an aggressive form of Crohn's disease. In another embodiment, treating the individual comprises performing on the individual a small-bowel resection, colectomy and/or colonic resection. In another embodiment, the presence of each genetic risk variant has an additive effect on rapidity of Crohn's disease progression from a relatively less severe case of Crohn's disease to a relatively more severe case of Crohn's disease. In another embodiment, the one or more genetic risk variants comprise SEQ. ID. NO.: 1, SEQ. ID. NO.: 2, SEQ. ID. NO.: 3, SEQ. ID. NO.: 4, SEQ. ID. NO.: 5 and/or SEQ. ID. NO.: 6. In another embodiment, the one or more genetic risk variants comprise SEQ. ID. NO.: 7, SEQ. ID. NO.: 8, SEQ. ID. NO.: 9, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, SEQ. ID. NO.: 18, SEQ. ID. NO.: 19, SEQ. ID. NO.: 20, SEQ. ID. NO.: 21, and/or SEQ. ID. NO.: 22. In another embodiment, the one or more genetic risk variants comprise SEQ. ID. NO.: 23, SEQ. ID. NO.: 24, SEQ. ID. NO.: 25, SEQ. ID. NO.: 26, SEQ. ID. NO.: 27, SEQ. ID. NO.: 28, SEQ. ID. NO.: 29, SEQ. ID. NO.: 30, SEQ. ID. NO.: 31, SEQ. ID. NO.: 32, SEQ. ID. NO.: 33, SEQ. ID. NO.: 34, SEQ. ID. NO.: 35, SEQ. ID. NO.: 36, SEQ. ID. NO.: 37, SEQ. ID. NO.: 38, SEQ. ID. NO.: 39, SEQ. ID. NO.: 40, SEQ. ID. NO.: 41, SEQ. ID. NO.: 42, SEQ. ID. NO.: 43, SEQ. ID. NO.: 44, SEQ. ID. NO.: 45, SEQ. ID. NO.: 46, SEQ. ID. NO.: 47, SEQ. ID. NO.: 48, SEQ. ID. NO.: 49, SEQ. ID. NO.: 50, SEQ. ID. NO.: 51, and/or SEQ. ID. NO.: 52. In another embodiment, the individual is a child 17 years old or younger.

Other embodiments include a method of diagnosing susceptibility to Crohn's disease in an individual, comprising obtaining a sample from the individual, assaying the sample for the presence or absence of one or more genetic risk variants, and diagnosing susceptibility to Crohn's disease in the individual based on the presence of one or more genetic risk variants, where the one or more genetic risk variants are located at the genetic loci of 8q24, 16p11, and/or Bromodomain and WD repeat domain containing 1 (BRWD1). In another embodiment, the one or more genetic risk variants comprise SEQ. ID. NO.: 1, SEQ. ID. NO.: 2, SEQ. ID. NO.: 3, SEQ. ID. NO.: 4, SEQ. ID. NO.: 5 and/or SEQ. ID. NO.: 6. In another embodiment, the one or more genetic risk variants comprise SEQ. ID. NO.: 7, SEQ. ID. NO.: 8, SEQ. ID. NO.: 9, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, SEQ. ID. NO.: 18, SEQ. ID. NO.: 19, SEQ. ID. NO.: 20, SEQ. ID. NO.: 21, and/or SEQ. ID. NO.: 22. In another embodiment, the one or more genetic risk variants comprise SEQ. ID. NO.: 23, SEQ. ID. NO.: 24, SEQ. ID. NO.: 25, SEQ. ID. NO.: 26, SEQ. ID. NO.: 27, SEQ. ID. NO.: 28, SEQ. ID. NO.: 29, SEQ. ID. NO.: 30, SEQ. ID. NO.: 31, SEQ. ID. NO.: 32, SEQ. ID. NO.: 33, SEQ. ID. NO.: 34, SEQ. ID. NO.: 35, SEQ. ID. NO.: 36, SEQ. ID. NO.: 37, SEQ. ID. NO.: 38, SEQ. ID. NO.: 39, SEQ. ID. NO.: 40, SEQ. ID. NO.: 41, SEQ. ID. NO.: 42, SEQ. ID. NO.: 43, SEQ. ID. NO.: 44, SEQ. ID. NO.: 45, SEQ. ID. NO.: 46, SEQ. ID. NO.: 47, SEQ. ID. NO.: 48, SEQ. ID. NO.: 49, SEQ. ID. NO.: 50, SEQ. ID. NO.: 51, and/or SEQ. ID. NO.: 52. In another embodiment, the individual is a child 17 years old or younger.

Other features and advantages of the invention will become apparent from the following detailed description, taken in conjunction with the accompanying drawings, which illustrate, by way of example, various embodiments of the invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“IBD” as used herein is an abbreviation of inflammatory bowel disease.

“CD” as used herein is an abbreviation of Crohn's Disease.

“UC” as used herein is an abbreviation of ulcerative colitis.

“ANCA” as used herein refers to anti-neutrophil cytoplasmic antibody.

As used herein, “SNP” means single nucleotide polymorphism.

“GWAS” as used herein is an abbreviation of genome wide associations.

“Antibody sum” as used herein refers to the number of positive antibody markers per individual.

“Antibody quartile score” as used herein refers to the quartile score for each antibody level.

“Quartile sum score” as used herein refers to the sum of quartile scores for all types of antibody tested.

“Complication” as used herein refers to a severe form of Crohn's disease that may be associated with an internal penetrating and/or stricturing disease phenotype, or conditions that require surgical procedures associated with the treatment of Crohn's disease due to unresponsiveness to non surgical treatments.

“Surgery” as used herein refers to a surgical procedure related to Inflammatory Bowel Disease or Crohn's disease, including small-bowel resections, colectomy and colonic resection.

“Progressive” Crohn's disease or “aggressive” Crohn's disease as used herein refers to a condition that may be characterized by the rapid progression from an uncomplicated to complicated phenotype in a Crohn's disease patient. Complicated phenotypes of Crohn's disease patients may include, for example, the development of internal penetrating, stricturing disease and/or perianal penetrating. This is in contrast to an uncomplicated phenotype that may be characterized, for example, by nonpenetrating and/or nonstricturing.

Various survival studies are described herein. The survival studies utilized a cohort at time of diagnosis of Crohn's disease (time zero) and then followed them forward to complication and/or surgery phenotypes, with time from diagnosis to complication and/or surgery measured in months. A genetic risk variant and/or risk marker with a 0.05 or less significance value in survival outcome is indicative of a statistically significant association with surgery and/or complication phenotype.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

As disclosed herein, the inventors examined 34 SNPs to look at the association with surgery in 173 pediatric patients with Crohn's Disease. The outcome was any Crohn's Disease surgery. Specifically, SNPs were found by multivariate analysis to be independently associated with surgery. Additionally, survival analysis was used to determine whether specific SNPs were associated with faster progression to surgery, where survival analysis as a predictive model showed that as patients were determined to have more of the significant genes, the progression to surgery was faster. Some of the genetic loci found to be significant include 8q24, 16p11, BRWD1 and TNFSF15.

As further disclosed herein, the inventors performed genome-wide association studies (GWAS) to determine the association between the presence of SNPs in an individual with Crohn's disease and the result of complication and/or surgery. Stepwise variable selection was then applied to logistic regression models (3 for complication and 5 for surgery) including SNPs selected from GWAS, gender, age, disease location, ANCA and antibody sum/quartile score as predictors. Survival analyses for complication and surgery were performed with the Cox Regression model. First, in order to select significant SNPs, genome-wide survival analyses were performed with a Cox regression model, in which each SNP was a predictor. Second, stepwise variable selection was applied to Cox regression models (3 models for complication and 5 models for surgery) using SNPs, gender, age, disease location, ANCA, and antibody sum/antibody quartile score as predictors. Third, the survival functions obtained by the Kaplan-Meier (KM) estimator among subgroups of patients were compared, which were subgrouped with 25% quartile and 75% quartile of the genetic risk score calculated from the selected model in the second step for each regression model (group 1 if risk score≤25% quartile, group 2 if 25% quartile<risk score<75% quartile, and group 3 if risk score>75% quartile). Finally, for each subgroup, the survival functions were compared across the models. For all 3 complication models, the survival functions obtained by the KM estimator were significantly different among subgroups of patients. For all 3 subgroups, the survival functions across the 3 models were statistically indistinguishable with a significance level of 0.05. As further disclosed herein, for all 5 surgery models, the survival functions obtained by the KM estimator were significantly different among subgroups of patients. For all 3 subgroups, the survival functions across the 5 models were statistically indistinguishable with a significance level of 0.05.

In one embodiment, the present invention provides a method of prognosing Crohn's Disease in an individual by determining the presence or absence of one or more risk factors, where the presence of one or more risk factors is indicative of an aggressive form of Crohn's Disease. In another embodiment, the aggressive form of Crohn's Disease is characterized by a fast progression from a relatively less severe form of Crohn's disease to a relatively more severe case of Crohn's disease. In another embodiment, the aggressive form of Crohn's Disease is characterized by conditions requiring surgical treatment associated with treating the Crohn's disease. In another embodiment, the one or more risk factors are described in Tables 1-6 herein. In another embodiment, the risk factors include one or more genetic and serological or demographic or disease location or disease behavior risk factors. In another embodiment the disease behavior risk factor is stricture or penetration. In another embodiment a serological risk factor is ASCA. In another embodiment the disease location risk factor is the ileal, colonic or ileocolonic form of Crohn's disease, or a combination thereof. In another embodiment the demographic risk factors are gender and/or age.

In another embodiment, the presence of each additional risk factor has an additive effect on the rate of progression. In another embodiment, the individual is a child 17 years old or younger.

In one embodiment, the present invention provides a method of diagnosing susceptibility to Crohn's Disease in an individual by determining the presence or absence of one or more risk factors described in Tables 1-6 herein, where the presence of one or more risk factors described in Tables 1-6 herein is indicative of susceptibility to Crohn's disease in the individual. In another embodiment, the risk factors include one or more genetic and serological or demographic or disease location or disease behavior risk factors. In another embodiment the disease behavior risk factor is stricture or penetration. In another embodiment a serological risk factor is ASCA. In another embodiment the disease location risk factor is the ileal, colonic or ileocolonic form of Crohn's disease, or a combination thereof. In another embodiment the demographic risk factors are gender and/or age. In another embodiment, the Crohn's Disease is associated with a complicated and/or conditions associated with the need for surgery phenotypes. In another embodiment, the individual is a child 17 years old or younger.

In another embodiment, the present invention provides a method of treating Crohn's Disease in an individual by determining the presence of one or more risk factors and treating the individual. In another embodiment, the one or more risk factors are described in Tables 1-6 herein. In another embodiment, the risk factors include one or more genetic and serological or demographic or disease location or disease behavior risk factors. In another embodiment the disease behavior risk factor is stricture or penetration. In another embodiment a serological risk factor is ASCA. In another embodiment the disease location risk factor is the ileal, colonic or ileocolonic form of Crohn's disease, or a combination thereof. In another embodiment, the demographic risk factors are gender and/or age. In another embodiment, the individual is a child.

A variety of methods can be used to determine the presence or absence of a variant allele or haplotype or serological profile. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature,” Nucleic Acids Research 28:655-661 (2000)). Minor grove binders include, but are not limited to, compounds such as dihydrocyclopyrroloindole tripeptide (DPI,).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention may be practiced using one or any combination of the well known assays described above or another art-recognized genetic assay.

Similarly, there are many techniques readily available in the field for detecting the presence or absence of serological markers, polypeptides or other biomarkers, including protein microarrays. For example, some of the detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Similarly, there are any number of techniques that may be employed to isolate and/or fractionate biomarkers. For example, a biomarker may be captured using biospecific capture reagents, such as antibodies, aptamers or antibodies that recognize the biomarker and modified forms of it. This method could also result in the capture of protein interactors that are bound to the proteins or that are otherwise recognized by antibodies and that, themselves, can be biomarkers. The biospecific capture reagents may also be bound to a solid phase. Then, the captured proteins can be detected by SELDI mass spectrometry or by eluting the proteins from the capture reagent and detecting the eluted proteins by traditional MALDI or by SELDI. One example of SELDI is called “affinity capture mass spectrometry,” or “Surface-Enhanced Affinity Capture” or “SEAC,” which involves the use of probes that have a material on the probe surface that captures analytes through a non-covalent affinity interaction (adsorption) between the material and the analyte. Some examples of mass spectrometers are time-of-flight, magnetic sector, quadrupole filter, ion trap, ion cyclotron resonance, electrostatic sector analyzer and hybrids of these.

Alternatively, for example, the presence of biomarkers such as polypeptides may be detected using traditional immunoassay techniques. Immunoassay requires biospecific capture reagents, such as antibodies, to capture the analytes. The assay may also be designed to specifically distinguish protein and modified forms of protein, which can be done by employing a sandwich assay in which one antibody captures more than one form and second, distinctly labeled antibodies, specifically bind, and provide distinct detection of, the various forms. Antibodies can be produced by immunizing animals with the biomolecules. Traditional immunoassays may also include sandwich immunoassays including ELISA or fluorescence-based immunoassays, as well as other enzyme immunoassays.

Prior to detection, biomarkers may also be fractionated to isolate them from other components in a solution or of blood that may interfere with detection. Fractionation may include platelet isolation from other blood components, sub-cellular fractionation of platelet components and/or fractionation of the desired biomarkers from other biomolecules found in platelets using techniques such as chromatography, affinity purification, 1D and 2D mapping, and other methodologies for purification known to those of skill in the art. In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Associations with Outcome of Surgery—Table 1

Using a GWAS top hits and using Crohn's Disease surgery as an outcome, 34 SNPs were tested to look at the association with surgery in 173 children. Table 1 lists five (5) SNPs that, out of the 34 initially tested, demonstrated the strongest association with the outcome of surgery when individually tested after the initial genome wide association analysis. The first column of Table 1 lists the SNPs, the second column lists the p-value of association, and the third column lists the odds ratio (95% confidence limits) for the increased risk of surgery for those patients with the minor allele in the respective gene.

TABLE 1

| | | |
|---|---|---|
| rs1551398 (8q24) | 0.0082 | 3.3 (1.36, 8.1) |
| rs1968752 (16p11) | 0.0044 | 0.32 (0.15, 0.69) |
| rs2836878 (21q22/BRWD1) | 0.08 | 0.5 (0.2, 1.1) |
| rs4574921 (TNFSF15) | 0.06 | 0.44 (0.2, 1.0) |
| rs8049439 (16p11) | 0.003 | 0.31 (0.15, 0.67) |

The third column in Table 1, or “risk factor” column, interprets the alleles in the context of the results deciphered and referenced in Tables 2-4 below. In Table 1, the results were rearranged so that each allele tested was the specific combination of alleles that increased risk. Note that in Table 1, some of the odds ratios were larger than 1, where for example rs1551398 the odds ratio is 3.3. For others the odds ratio were less than 1, such as for example rs1969752 where the risk is 0.32. An odds ratio of less than 1 means that the particular test is showing a decreased risk, such as in this case a decreased risk for the minor allele. These were re-arranged so that each SNP would be showing an increase in risk. A decreased risk for the minor allele would mean an increased risk for the major allele.

Finally, all of the SNPs were put into a single statistical model and tested together, with the result being that four of the SNPs remained significant while the rs8049439 SNP does not remain in the model. This is not a surprising result given that rs8049439 is in the same gene as the SNP rs1968752. Each is significant when tested individually, but only one is needed when these are tested together.

### Example 2

### Multivariate Analysis Demonstrated 4 SNPs Independently Associated with Surgery Outcome—Table 2

Table 2 describes multivariate analysis demonstrating the four SNPs referenced below as independently associated with surgery outcome. For example in Table 2 below, for rs1551398_2c, the presence of “12” or “22” increases the likelihood of requiring surgery in the individual by 1.18 with a significance of 0.121. The alleles are referenced in Table 6 below, where for example, the presence of the minor allele (which is “G” if using the top strand, and “C” if using the forward strand), increases the likelihood for surgery by 1.18. Similarly, for example in Table 2 below, for rs1968752, an individual homozygous for the major allele (or “A” for both top and forward strand) increases the likelihood of surgery by 1.2 with a significance of 0.0035. Table 2 uses an estimation of the maximum likelihood of the effect.

TABLE 2
Analysis of Maximum Likelihood Estimates

| | | | Standard | Wald | |
|---|---|---|---|---|---|
| Parameter | DF | Estimate | Error | Chi-Square | Pr > ChiSq |
| Intercept | 1 | −4.1426 | 0.697 | 35.3235 | <.0001 |
| rs1551398_2c | 1 | 1.1807 | 0.4705 | 6.2983 | 0.0121 |
| (12/22 vs. 11) | | | | | |
| rs1968752_11 | 1 | 1.2173 | 0.4169 | 8.525 | 0.0035 |
| (11 vs. 12/22) | | | | | |
| rs2836878_11 | 1 | 0.8441 | 0.4291 | 3.8697 | 0.0492 |
| (11 vs. 12/22) | | | | | |
| rs4574921_11 | 1 | 1.119 | 0.4726 | 5.6071 | 0.0179 |
| (11 vs. 12/22) | | | | | |

### Example 3

### Odds Ratio Estimates—Table 3

Table 3 demonstrates how the risk factors may increase the odds ratio (compared to Table 2 above which is estimating likelihood) for going to surgery using the Wald test. For example, a subject having the presence of the minor allele for rs1551398 has an odds ratio of requiring surgery of 3.2.

TABLE 3

| | | |
|---|---|---|
| | | 95% Wald Confidence |
| Effect | Point Estimate | Limits |

| | | | |
|---|---|---|---|
| Rs1551398 | 3.257 | 1.295 | 8.189 |
| Rs1968752_11 | 3.378 | 1.492 | 7.649 |
| Rs2836878 | 2.326 | 1.003 | 5.393 |
| Rs4574921_11 | 3.062 | 1.213 | 7.731 |

### Example 4

### Survival Analysis for Time to Surgery—Table 4

Table 4 below describes the use of survival analysis to determine whether certain SNPs were associated with faster progression to Crohn's Disease surgery. The common allele is designated as “1”, and the rare allele is designated as “2.”

TABLE 4

| | | | | | | | | | |
|---|---|---|---|---|---|---|---|---|---|
| rs1968752 | 11 | 62 | 12 | 50 | 80.65 | Log-Rank | 0.0177 | 0.37 | 0.02 |
| | 12/22 | 117 | 9 | 108 | 92.31 | Wilcoxon | 0.0118 | (12/22 vs. 11) | |
| rs8049439 | 11 | 66 | 13 | 53 | 80.3 | Log-Rank | 0.004 | 0.3 | 0.008 |
| | 12/22 | 113 | 8 | 105 | 92.92 | Wilcoxon | 0.0113 | (12/22 vs. 11) | |
| rs11174631 | 11 | 154 | 14 | 140 | 90.91 | Log-Rank | 0.0319 | 2.6 | 0.04 |
| | 12/22 | 25 | 7 | 18 | 72 | Wilcoxon | 0.5321 | (12/22 vs. 11) | |

### Example 5

### Survival Analysis Predictive Model—Table 5

Table 5 below uses survival analysis regarding the question of whether risk factors are counted, does the patient progress to surgery faster. The risk factor column is the count of the risk alleles referenced in Table 6 below; the overall significance is shown in the right most column. The total shows how many subjects had risk alleles; failed is the number that required surgery; censored is the number that did not require surgery but that had the date when they were last known to not have surgery. As demonstrated below, survival analysis as a predictive model showed that as patients had more genes, then the progression to surgery was faster (0 vs. 4 genes). The four (4) genes were the same as those found in the multivariate analysis referenced above.

TABLE 5

| riskfactor | total | failed | censored | % censored | logrank |
|---|---|---|---|---|---|
| 0 | 10 | 0 | 10 | 100% | <0.0001 |
| 1 | 36 | 0 | 36 | 100% | |
| 2 | 79 | 10 | 69 | 87% | |
| 3 | 43 | 6 | 37 | 86% | |
| 4 | 11 | 5 | 6 | 54% | |

### Example 6

### Corresponding Alleles for Six (6) SNPs Referenced Herein—Table 6

Table 6 describes the referenced alleles for the listed SNPs, where the top strand designates the actual allele used in the analysis herein, and the forward strand designates the same allele on the reference genome assembly number 36 as referenced in the National Center for Biotechnology Information (NCBI).

TABLE 6

| | Top Strand | | Forward Strand | | |
|---|---|---|---|---|---|
| | Minor | Major | (dbsnp) | | |
| | Allele | Allele | Minor | Major | |
| SNPid | (“2”) | (“1”) | Allele | Allele | Risk Factor |
| rs1551398 | G | A | C | T | Presence of minor |
| (SEQ. ID. | | | | | allele |
| NO.: 1) | | | | | |
| rs1968752 | A | C | A | C | Homozygous for |
| (SEQ. ID. | | | | | major allele |
| NO.: 2) | | | | | |
| rs2836878 | A | G | A | G | Homozygous for |
| (SEQ. ID. | | | | | major allele |
| NO.: 3) | | | | | |
| rs4574921 | G | A | C | T | Homozygous for |
| (SEQ. ID. | | | | | major allele |
| NO.: 4) | | | | | |
| rs8049439 | G | A | C | T | Presence of minor |
| (SEQ. ID. | | | | | allele |
| NO.: 5) | | | | | |
| rs11174631 | A | G | C | T | Presence of minor |
| (SEQ. ID. | | | | | allele |
| NO.: 6) | | | | | |

### Example 7

### Additional Genome-Wide Association Studies

Genome-wide association studies (GWAS) were performed to determine the association between disease phenotypes (complication and surgery) and single nucleotide polymorphisms (SNPs). Then, stepwise variable selection was applied to logistic regression models (3 models for complication and 5 models for surgery) incorporating: SNPs selected from GWAS, gender, age, disease location, ANCA and antibody sum/antibody quartile score as predictors.

### Example 8

### Significant SAT's (p<5×10⁻⁵) Selected from GWAS with Complication

For complication, Table 7 shows 16 SNPs with p-values less than 5×10⁻⁵ were selected throughout the GWAS. SNPs rs7181301, rs11223560, rs2245872, rs261827, rs12909385, rs4787664, rs11009506, rs7672594, rs1781873, rs17771939, rs10180293, rs4833624, rs12512646, rs6413435, rs1889926, and rs4305427 are described herein as SEQ. ID. NOS.: 7-22, respectively.

TABLE 7
List of Significant SNPs (p < 5 × 10⁻⁵) selected from GWAS with Complication

| Obs | CHR | SNP | BP | OR | STAT | P |
|---|---|---|---|---|---|---|
| 1 | 15 | rs7181301 | 96440815 | 3.2440 | 4.662 | .000003137 |
| 2 | 11 | rs11223560 | 133066609 | 1.9330 | 4.374 | .000012180 |
| 3 | 1 | rs2245872 | 37704373 | 1.9750 | 4.347 | .000013810 |
| 4 | 1 | rs261827 | 239136994 | 1.9660 | 4.318 | .000015730 |
| 5 | 15 | rs12909385 | 55484367 | 2.0650 | 4.238 | .000022590 |
| 6 | 16 | rs4787664 | 23958740 | 0.3960 | −4.234 | .000022940 |
| 7 | 10 | rs11009506 | 34063503 | 0.4937 | −4.223 | .000024150 |
| 8 | 4 | rs7672594 | 120467991 | 1.9350 | 4.206 | .000026030 |
| 9 | 19 | rs1781873 | 21269271 | 0.5245 | −4.204 | .000026230 |
| 10 | 8 | rs17771939 | 94328281 | 0.4497 | −4.103 | .000040850 |
| 11 | 2 | rs10180293 | 206330821 | 0.3500 | −4.100 | .000041300 |
| 12 | 4 | rs4833624 | 120804945 | 1.9030 | 4.097 | .000041890 |
| 13 | 4 | rs12512646 | 120805181 | 1.9030 | 4.097 | .000041890 |
| 14 | 19 | rs6413435 | 18358137 | 2.1750 | 4.094 | .000042490 |
| 15 | 1 | rs1889926 | 65470767 | 2.0270 | 4.093 | .000042620 |
| 16 | 3 | rs4305427 | 68750047 | 1.8530 | 4.075 | .000045970 |

### Example 9

### Selection of 3 Logistic Regression Models

Next, 3 logistic regression models were considered in order to measure the strength of association between the response of complication (Yes/No) and the predictors. The first model included: 16 SNPs, gender, age, and disease location. The second model included: 16 SNPs, gender, age, disease location, ANCA, and antibody quartile score. The third model included: 16 SNPs, gender, age, disease location, ANCA, and antibody sum. After stepwise variable selection, primary associations with complication were determined.

### Example 10

### Model 1: Logistic Regression of Complication with 16 SNPs Selected, Sex1, Age, and Sb1

As indicated in Table 8, in the first model, 14 out of 16 SNPs, gender, age and disease location were determined to be statistically significant.

TABLE 8a
Analysis of Maximum Likelihood Estimates

| | | | Standard | Wald | |
|---|---|---|---|---|---|
| Parameter | DF | Estimate | Error | Chi-Square | Pr > ChiSq |
| rs7181301 | 1 | 1.1091 | 0.3011 | 13.5657 | 0.0002 |
| rs11223560 | 1 | 0.0536 | 0.2382 | 12.8386 | 0.0003 |
| rs2245872 | 1 | 0.6269 | 0.2085 | 9.0386 | 0.0026 |
| rs261827 | 1 | −0.7731 | 0.3323 | 5.4136 | 0.0200 |
| rs12909385 | 1 | −0.8385 | 0.2790 | 9.0297 | 0.0027 |
| rs11009506 | 1 | −0.6072 | 0.2039 | 0.8695 | 0.0029 |
| rs1781873 | 1 | 0.8734 | 0.2439 | 12.8222 | 0.0003 |
| rs17771939 | 1 | −0.7792 | 0.2309 | 11.3921 | 0.0007 |
| rs10180293 | 1 | 0.9107 | 0.2031 | 20.1041 | <.0001 |
| rs4833624 | 1 | 0.5907 | 0.2298 | 6.6096 | 0.0101 |
| rs12512646 | 1 | −1.8591 | 0.3335 | 31.0658 | <.0001 |
| rs6413435 | 1 | −0.8896 | 0.2771 | 10.3050 | 0.0013 |
| rs1889926 | 1 | −0.6911 | 0.2471 | 7.8193 | 0.0052 |
| rs4305427 | 1 | 1.3481 | 0.4186 | 10.3705 | 0.0013 |
| sex1 | 1 | −0.8994 | 0.2913 | 9.5327 | 0.0020 |
| age_at_dx2 | 1 | 1.0368 | 0.2977 | 12.1312 | 0.0005 |
| sb1 | 1 | 1.2903 | 0.3765 | 11.7450 | 0.0006 |

Hosmer and Lemeshow Goodness-of-Fit Test
AUC = 0.906

| Chi-Square | DF | Pr > ChiSq |
|---|---|---|
| 3.5183 | 8 | 0.8378 |

TABLE 8b
Odds Ratio Estimates

| | | |
|---|---|---|
| | Point | 95% Wald |
| Effect | Estimate | Confidence Limits |

| | | | |
|---|---|---|---|
| rs7181301 | 3.032 | 1.680 | 5.470 |
| rs11223560 | 2.348 | 1.472 | 3.745 |
| rs2245872 | 1.072 | 1.244 | 2.817 |
| rs261827 | 0.462 | 0.241 | 0.885 |
| rs12909385 | 0.432 | 0.250 | 0.747 |
| rs11009506 | 0.545 | 0.365 | 0.813 |
| rs1781873 | 2.395 | 1.485 | 3.863 |
| rs17771939 | 0.459 | 0.292 | 0.721 |
| rs10100293 | 2.486 | 1.670 | 3.702 |
| rs4833624 | 1.805 | 1.151 | 2.832 |
| rs12512646 | 0.156 | 0.081 | 0.300 |
| rs6413435 | 0.411 | 0.239 | 0.707 |
| rs1889926 | 0.501 | 0.309 | 0.813 |
| rs4305427 | 3.850 | 1.695 | 8.745 |
| sex1 | 0.407 | 0.230 | 0.720 |
| age_at_dx2 | 2.820 | 1.574 | 5.054 |
| sb1 | 3.634 | 1.737 | 7.60 |

### Example 11

### Model 2: Logistic Regression of Complication with 16 SNPs Selected, Sex1, Age at Diagnosis, Sb1, Anca p1, and Antibody Quartile

As indicated in Table 9, in the second model, 14 out of 16 SNPs, gender, age, disease location, ANCA, and antibody quartile score were determined to be statistically significant.

TABLE 9a
Analysis of Maximum Likelihood Estimates

| | | | Standard | Wald | |
|---|---|---|---|---|---|
| Parameter | DF | Estimate | Error | Chi-Square | Pr > ChiSq |
| rs7181381 | 1 | 0.9923 | 0.3242 | 9.3684 | 0.0022 |
| rs11223560 | 1 | 0.8874 | 0.2577 | 11.8598 | 0.0006 |
| rs2245872 | 1 | 0.6265 | 0.2358 | 7.1581 | 0.0075 |
| rs261827 | 1 | −0.7985 | 0.3761 | 4.5083 | 0.0337 |
| rs12909385 | 1 | −1.1616 | 0.3098 | 14.1305 | 0.0002 |
| rs11009586 | 1 | −0.8349 | 0.2349 | 12.6354 | 0.0004 |
| rs1781873 | 1 | 0.9181 | 0.2639 | 11.8927 | 0.0006 |
| rs17771939 | 1 | −0.8549 | 0.2465 | 12.0254 | 0.0005 |
| rs10188293 | 1 | 1.0455 | 0.2291 | 20.0239 | <.0001 |
| rs4833624 | 1 | 0.6598 | 0.2565 | 6.6143 | 0.0101 |
| rs12512646 | 1 | −2.1169 | 0.3715 | 32.4764 | <.0001 |
| rs6413435 | 1 | −0.9961 | 0.3021 | 10.8723 | 0.0010 |
| rs1889926 | 1 | −0.8970 | 0.2768 | 10.5001 | 0.0012 |
| rs4385427 | 1 | 1.1535 | 0.4372 | 6.9619 | 0.0083 |
| sex1 | 1 | −0.9212 | 0.3193 | 8.3234 | 0.0039 |
| age_at_dx2 | 1 | 1.0503 | 0.3278 | 10.4647 | 0.0012 |
| anca_P1 | 1 | −1.5651 | 0.4747 | 10.8730 | 0.0010 |
| ab_quar1 | 1 | 1.0654 | 0.1933 | 30.3832 | <.0001 |

Hosmer and Lemeshow Goodness-of-Fit Test
AUC = 0.930

| Chi-Square | DF | Pr > ChiSq |
|---|---|---|
| 7.1251 | 8 | 0.5232 |

TABLE 9b
Odds Ratio Estimates

| | | |
|---|---|---|
| | Point | 95% Wald |
| Effect | Estimate | Confidence Limits |

| | | | |
|---|---|---|---|
| rs7181381 | 2.697 | 1.429 | 5.892 |
| rs11223588 | 2.429 | 1.466 | 4.825 |
| rs2245872 | 1.875 | 1.183 | 2.972 |
| rs281827 | 0.450 | 0.215 | 0.940 |
| rs12989385 | 0.313 | 0.171 | 0.574 |
| rs11009506 | 0.434 | 0.274 | 0.688 |
| rs1701873 | 2.485 | 1.481 | 4.168 |
| rs17771939 | 0.425 | 0.262 | 0.690 |
| rs10186293 | 2.845 | 1.816 | 4.457 |
| rs4833624 | 1.934 | 1.178 | 3.198 |
| rs12512646 | 0.120 | 0.058 | 0.243 |
| rs6413435 | 0.369 | 0.284 | 0.668 |
| rs1889925 | 0.408 | 0.237 | 0.702 |
| rs4385427 | 3.169 | 1.345 | 7.466 |
| sex1 | 0.398 | 0.213 | 0.744 |
| age_at_dx2 | 2.887 | 1.519 | 5.488 |
| anca_P1 | 0.289 | 0.082 | 0.530 |
| ab_quar1 | 2.902 | 1.987 | 4.239 |

### Example 12

### Model 3: Logistic Regression of Complication with 16 SNPs Selected, Sex1, Age at Diagnosis, Sb1, Anca p1, and Antibody Sum

As indicated in Table 10, in the third model, 14 out of 16 SNPs, gender, age, disease location, ANCA, and antibody sum were determined to be statistically significant.

TABLE 10a
Analysis of Maximum Likelihood Estimates

| | | | Standard | Wald | |
|---|---|---|---|---|---|
| Parameter | DF | Estimate | Error | Chi-Square | Pr > ChiSq |
| rs7181381 | 1 | 1.0739 | 0.3277 | 10.7356 | 0.0011 |
| rs11223560 | 1 | 0.8708 | 0.2568 | 11.5812 | 0.0007 |
| rs2245872 | 1 | 0.6764 | 0.2316 | 0.5768 | 0.0034 |
| rs261827 | 1 | −0.6401 | 0.3668 | 3.8462 | 0.0009 |
| rs12909385 | 1 | −1.0195 | 0.3878 | 11.0258 | 0.0009 |
| rs11009586 | 1 | −0.6543 | 0.2283 | 0.2149 | 0.0042 |
| rs1761873 | 1 | 0.8869 | 0.2617 | 11.5338 | 0.0007 |
| rs17771939 | 1 | −0.8878 | 0.2486 | 12.7512 | 0.0004 |
| rs10180293 | 1 | 1.0645 | 0.2298 | 21.4536 | <.0001 |
| rs4833624 | 1 | 0.7220 | 0.2579 | 7.8399 | 0.0051 |
| rs12512646 | 1 | −1.8675 | 0.3693 | 25.5759 | <.0001 |
| rs6413435 | 1 | −0.8736 | 0.3822 | 0.3581 | 0.0038 |
| rs1889926 | 1 | −0.7832 | 0.2717 | 0.3072 | 0.0039 |
| rs4305427 | 1 | 1.1488 | 0.4495 | 0.5386 | 0.0106 |
| sex1 | 1 | −0.8954 | 0.3206 | 7.7986 | 0.0052 |
| age_at_dx2 | 1 | 1.0866 | 0.3278 | 9.4278 | 0.0021 |
| sb1 | 1 | 0.8180 | 0.4864 | 4.6514 | 0.0441 |
| anca_P1 | 1 | −1.3505 | 0.4672 | 0.3542 | 0.0038 |
| ab_sum | 1 | 0.6831 | 0.1412 | 23.4165 | <.0001 |

Hosmer and Lemeshow Goodness-of-Fit Test
AUC = 0.929

| Chi-Square | DF | Pr > ChiSq |
|---|---|---|
| 4.9462 | 8 | 0.7633 |

TABLE 10b
Odds Ratio Estimates

| | | |
|---|---|---|
| | Point | 95% Wald |
| Effect | Estimate | Confidence Limits |

| | | | |
|---|---|---|---|
| rs7181301 | 2.927 | 1.540 | 5.564 |
| rs11223560 | 2.389 | 1.444 | 3.952 |
| rs2245872 | 1.971 | 1.252 | 3.103 |
| rs261827 | 0.527 | 0.257 | 1.082 |
| rs12909385 | 0.361 | 0.198 | 0.659 |
| rs11009506 | 0.520 | 0.332 | 0.813 |
| rs1781873 | 2.432 | 1.456 | 4.063 |
| rs17771939 | 0.412 | 0.253 | 0.670 |
| rs10180293 | 2.899 | 1.848 | 4.549 |
| rs4833624 | 2.053 | 1.242 | 3.412 |
| rs12512646 | 0.155 | 0.075 | 0.319 |
| rs6413435 | 0.417 | 0.231 | 0.755 |
| rs1883926 | 0.457 | 0.268 | 0.778 |
| rs4305427 | 3.154 | 1.307 | 7.613 |
| sex1 | 0.408 | 0.218 | 0.766 |
| age_at_dx2 | 2.736 | 1.439 | 5.203 |
| sb1 | 2.266 | 1.022 | 5.026 |
| anca_P1 | 0.259 | 0.104 | 0.647 |
| ab_sum | 1.980 | 1.501 | 2.611 |

### Example 13

### Significant SNPs (p<5×10⁻⁵) Selected from GWAS with Surgery

As indicated in Table 11, for surgery, 30 significant SNPs were selected with p-values less than 5×10⁻⁵. SNPs rs6491069, rs12100242, rs7575216, rs9742643, rs7333546, rs10825455, rs187783, rs261804, rs501691, rs2993493, rs1749969, rs7157738, rs1325607, rs2018454, rs1403146, rs261827, rs487675, rs12386815, rs2928686, rs1168566, rs2698174, rs16842384, rs705308, rs12909385, rs724685, rs9864383, rs11845504, rs898716, rs7181301, and rs913735 are described herein as SEQ. ID. NOS.: 23-52, respectively.

TABLE 11
List of Significant SNPs (p < 5 × 10⁻⁵) selected from GWAS with Surgery

| Obs | CHR | snp | BP | OR | STAT | P |
|---|---|---|---|---|---|---|
| 1 | 13 | rs6491069 | 25050039 | 2.6550 | 4.805 | .000001545 |
| 2 | 13 | rs12100242 | 25078845 | 2.5750 | 4.712 | .000002456 |
| 3 | 2 | rs7575216 | 39257914 | 3.3980 | 4.683 | .000002832 |
| 4 | 13 | rs9742643 | 25026096 | 2.6140 | 4.681 | .000002857 |
| 5 | 13 | rs7333546 | 24949574 | 2.4770 | 4.587 | .000004506 |
| 6 | 10 | rs10825455 | 56496449 | 3.6210 | 4.530 | .000005886 |
| 7 | 1 | rs187783 | 239119745 | 2.0080 | 4.530 | .0000058 [symbol] |
| 8 | 1 | rs261804 | 239134094 | 1.9980 | 4.510 | .000006489 |
| 9 | 1 | rs501691 | 65516415 | 2.4290 | 4.505 | .000006628 |
| 10 | 1 | rs2993493 | 3010106 | 2.3910 | 4.476 | .000007605 |
| 11 | 1 | rs1749969 | 65500587 | 2.4150 | 4.468 | .000007886 |
| 12 | 14 | rs7157738 | 37944754 | 0.2567 | -4.457 | .000008296 |
| 13 | 1 | rs1325607 | 65523648 | 2.3660 | 4.445 | .000008792 |
| 14 | 19 | rs2018454 | 15873612 | 2.2490 | 4.390 | .000011360 |
| 15 | 3 | rs1403146 | 669 [symbol] 880 | 0.4707 | -4.371 | .000012380 |
| 16 | 1 | rs261827 | 239136994 | 1.9480 | 4.234 | .000022960 |
| 17 | 1 | rs487675 | 183067688 | 0.4671 | -4.188 | .000028120 |
| 18 | 8 | rs12386815 | 136027851 | 2.0230 | 4.173 | .000030130 |
| 19 | 8 | rs2928686 | 23477641 | 1.9670 | 4.165 | .000031180 |
| 20 | 14 | rs1168566 | 37957632 | 0.3417 | -4.151 | .000033170 |
| 21 | 18 | rs2698174 | 66897090 | 2.8540 | 4.149 | .000033390 |
| 22 | 2 | rs16842384 | 209650323 | 1.9410 | 4.145 | .000033940 |
| 23 | 7 | rs705308 | 97533299 | 0.4995 | -4.135 | .000035480 |
| 24 | 15 | rs12909385 | 55484367 | 2.0620 | 4.119 | .000038000 |
| 25 | 1 | rs724685 | 65499104 | 2.1800 | 4.118 | .000038200 |
| 26 | 3 | rs9864383 | 113264489 | 1.8730 | 4.115 | .000038780 |
| 27 | 14 | rs11845504 | 37965784 | 0.3454 | -4.121 | .000039470 |
| 28 | 10 | rs898716 | 14165659 | 2.0110 | 4.099 | .000041430 |
| 29 | 15 | rs7181301 | 96440815 | 2.7270 | 4.091 | .000043000 |
| 30 | 14 | rs913735 | 37951124 | 0.3393 | -4.072 | .000046680 |

[symbol]

indicates data missing or illegible when filed

Five logistic regression models with the response of surgery (Yes/No) and the predictors were considered. In the first model, the following variables were included: 30 SNPs, gender, age, and disease location. In the second model, the following variables were included: 30 SNPs, gender, age, disease location, ANCA, and antibody quartile score. In the third model, the following variables were included: 30 SNPs, gender, age, disease location, ANCA, antibody quartile score, internal penetrating, and stricture. In the fourth model, the following variables were included 16 SNPs, gender, age, disease location, ANCA, and antibody quartile score. In the fifth model, the following variables were included: 16 SNPs, gender, age, disease location, ANCA, antibody quartile score, internal penetrating, and stricture. After applying stepwise variable selection, primary associations with the response variable, surgery, were determined.

### Example 14

### Model 1: Logistic Regression of Surgery with 30 SNPs Selected, Sex1, Age at Diagnosis 2, and Sb1

As indicated in Table 12, in the first model, 17 out of 30 SNPs, and disease location were statistically significant.

TABLE 12a
Analysis of Maximum Likelihood Estimates

| | | | Standard | Wald | |
|---|---|---|---|---|---|
| Parameter | DF | Estimate | Error | Chi-Square | Pr > ChiSq |
| Intercept | 1 | 5.0724 | 2.3025 | 4.8532 | 0.0276 |
| rs9742643 | 1 | 1.0303 | 0.2833 | 13.2306 | 0.0003 |
| rs10825455 | 1 | −0.7561 | 0.2518 | 9.0209 | 0.0027 |
| rs261804 | 1 | 1.0697 | 0.2238 | 22.8398 | <.0001 |
| rs2993493 | 1 | −0.7851 | 0.4032 | 3.7918 | 0.0515 |
| rs1749969 | 1 | −0.9655 | 0.3172 | 9.2655 | 0.0023 |
| rs1325607 | 1 | −1.1166 | 0.3855 | 8.3903 | 0.0038 |
| rs1403146 | 1 | −0.9719 | 0.2404 | 16.3451 | <.0001 |
| rs261827 | 1 | −1.0055 | 0.2567 | 15.3366 | <.0001 |
| rs487675 | 1 | −0.3229 | 0.2425 | 11.5155 | 0.0007 |
| rs12386815 | 1 | −0.9665 | 0.3995 | 5.8525 | 0.0156 |
| rs16842384 | 1 | −0.9109 | 0.2991 | 9.2727 | 0.0023 |
| rs705308 | 1 | 3.3659 | 0.8530 | 15.3910 | <.0001 |
| rs12909385 | 1 | 1.1371 | 0.6592 | 2.9750 | 0.0846 |
| rs11845504 | 1 | −0.7177 | 0.2545 | 7.9539 | 0.0048 |
| rs898716 | 1 | −1.4424 | 0.4229 | 11.6328 | 0.0006 |
| rs7181301 | 1 | 1.4879 | 0.3961 | 14.1106 | 0.0002 |
| rs913735 | 1 | −0.6918 | 0.2729 | 6.4266 | 0.0112 |
| sb1 | 1 | 1.7672 | 0.4093 | 18.6413 | <.0001 |

Hosmer and Lemeshow Goodness-of-Fit Test

| Chi-Square | DF | Pr > ChiSq | |
|---|---|---|---|
| 5.6000 | 8 | 0.6919 | AUC = 0.925 |

TABLE 12b
Odds Ratio Estimates

| | |
|---|---|
| Point | 95% Wald |

| Effect | Estimate | Confidence | Limits |
|---|---|---|---|
| rs9742643 | 2.802 | 1.608 | 4.882 |
| rs10325455 | 0.469 | 0.287 | 0.769 |
| rs261804 | 2.915 | 1.880 | 4.528 |
| rs2933493 | 0.456 | 0.207 | 1.885 |
| rs1749969 | 0.381 | 0.205 | 0.789 |
| rs1325607 | 0.327 | 0.154 | 0.697 |
| rs1403146 | 0.373 | 0.236 | 0.686 |
| rs261827 | 0.366 | 0.221 | 0.685 |
| rs487675 | 0.439 | 0.273 | 0.786 |
| rs12386815 | 0.388 | 0.174 | 0.832 |
| rs16842384 | 0.402 | 0.224 | 0.723 |
| rs705303 | 28.959 | 5.389 | 155.623 |
| rs12909385 | 3.118 | 0.856 | 11.358 |
| rs11845504 | 0.468 | 0.296 | 0.803 |
| rs898716 | 0.236 | 0.103 | 0.541 |
| rs7181301 | 4.428 | 2.037 | 0.623 |
| rs913735 | 0.501 | 0.293 | 0.855 |
| sb1 | 5.855 | 2.625 | 13.059 |

### Example 15

### Model 2: Logistic Regression of Surgery with 30 SNPs Selected, Sex1, Age at Diagnosis2, Sb1, Anca p1 and Antibody Quartile 1

As indicated in Table 13, in the second model, 16 out of 30 SNPs, disease location, ANCA, and antibody quartile score were statistically significant.

TABLE 13a
Analysis of Maximum Likelihood Estimates

| | | | Standard | Wald | |
|---|---|---|---|---|---|
| Parameter | DF | Estimate | Error | Chi-Square | Pr > ChiSq |
| Intercept | 1 | 0.3430 | 2.0602 | 16.3997 | <.0001 |
| rs12100242 | 1 | −1.0226 | 0.2973 | 11.8330 | 0.0005 |
| rs10825455 | 1 | −1.0556 | 0.2856 | 13.6590 | 0.0002 |
| rs261804 | 1 | 0.6613 | 0.3033 | 4.7525 | 0.0293 |
| rs501691 | 1 | 0.5934 | 0.3249 | 3.3363 | 0.0670 |
| rs2993493 | 1 | −1.0127 | 0.4429 | 5.2278 | 0.0222 |
| rs1749969 | 1 | −1.0052 | 0.3479 | 8.3499 | 0.0039 |
| rs1325607 | 1 | −1.2141 | 0.4225 | 8.2570 | 0.0041 |
| rs1403146 | 1 | −0.9187 | 0.2563 | 12.8481 | 0.0003 |
| rs261827 | 1 | −1.1034 | 0.2752 | 16.0814 | <.0001 |
| rs487675 | 1 | −0.9426 | 0.2628 | 12.0659 | 0.0003 |
| rs12386815 | 1 | −1.1928 | 0.4211 | 8.0232 | 0.0046 |
| rs2698174 | 1 | −1.2826 | 0.3178 | 16.2873 | <.0001 |
| rs705308 | 1 | 2.0876 | 0.4645 | 20.2015 | <.0001 |
| rs898716 | 1 | −1.2787 | 0.4520 | 8.0030 | 0.0047 |
| rs7181301 | 1 | 1.2469 | 0.4273 | 8.5133 | 0.0035 |
| rs913735 | 1 | −0.6716 | 0.2966 | 5.1255 | 0.0236 |
| sb1 | 1 | 1.4063 | 0.4483 | 10.2042 | 0.0014 |
| anca_P1 | 1 | −0.9295 | 0.4477 | 4.3101 | 0.0379 |
| ab_quar1 | 1 | 0.0798 | 0.2059 | 18.2549 | <.0001 |

Hosmer and Lemeshow Goodness-of-Fit Test

| Chi-Square | DF | Pr > ChiSq | |
|---|---|---|---|
| 2.6755 | 8 | 0.9530 | AUC = 0.940 |

TABLE 13b
Odds Ratio Estimates

| | |
|---|---|
| Point | 95% Wald |

| Effect | Estimate | Confidence | Limits |
|---|---|---|---|
| rs12100242 | 0.360 | 0.201 | 0.644 |
| rs10825455 | 0.348 | 0.199 | 0.609 |
| rs261804 | 1.937 | 0.069 | 3.511 |
| rs501691 | 1.810 | 0.958 | 3.422 |
| rs2993493 | 0.363 | 0.152 | 0.865 |
| rs1749969 | 0.366 | 0.185 | 0.724 |
| rs1325607 | 0.297 | 0.130 | 0.680 |
| rs1403146 | 0.399 | 0.241 | 0.659 |
| rs261827 | 0.332 | 0.193 | 0.569 |
| rs487675 | 0.398 | 0.233 | 0.652 |
| rs12386815 | 0.383 | 0.133 | 0.693 |
| rs2698174 | 0.277 | 0.149 | 0.517 |
| rs785308 | 8.065 | 3.245 | 20.044 |
| rs898716 | 0.278 | 0.115 | 0.675 |
| rs7181301 | 3.479 | 1.506 | 0.040 |
| rs913735 | 0.511 | 0.286 | 0.914 |
| sb1 | 4.081 | 1.722 | 9.672 |
| anca_P1 | 0.395 | 0.164 | 0.949 |
| ab_quar1 | 2.410 | 1.610 | 3.609 |

### Example 16

### Model 3: Logistic Regression of Surgery with 30 SNPs Selected, Sex1, Age at Diagnosis2, Sb1, Anca p1, Antibody Quartile 1, Stricture 1, and Ip1

As demonstrated in Table 14, in the third model, 15 out of 30 SNPs, antibody quartile score, internal penetrating, and stricture were statistically significant.

TABLE 14a
Analysis of Maximum Likelihood Estimates

| | | | Standard | Wald | |
|---|---|---|---|---|---|
| Parameter | DF | Estimate | Error | Chi-Square | Pr > ChiSq |
| Intercept | 1 | 4.9758 | 2.4784 | 4.0307 | 0.0447 |
| rs6491069 | 1 | 2.1774 | 1.0160 | 4.5930 | 0.0321 |
| rs7575216 | 1 | −3.0946 | 1.2437 | 6.1916 | 0.0120 |
| rs10825455 | 1 | −1.0364 | 0.3235 | 10.2636 | 0.0014 |
| rs261804 | 1 | 0.8382 | 0.2606 | 10.3478 | 0.0013 |
| rs2993493 | 1 | −0.9862 | 0.4897 | 4.0558 | 0.0440 |
| rs1749969 | 1 | −1.0281 | 0.3993 | 6.6304 | 0.0100 |
| rs1325607 | 1 | −1.0502 | 0.4859 | 4.6724 | 0.0307 |
| rs1403146 | 1 | −0.3196 | 0.2898 | 0.0009 | 0.0047 |
| rs261827 | 1 | −1.0228 | 0.3157 | 10.4969 | 0.0012 |
| rs487675 | 1 | −0.9786 | 0.2799 | 12.2197 | 0.0005 |
| rs12386815 | 1 | −0.9141 | 0.4571 | 3.9993 | 0.0455 |
| rs2698174 | 1 | −1.2727 | 0.3486 | 13.3267 | 0.0003 |
| rs705308 | 1 | 2.3357 | 0.5514 | 17.9452 | <.0001 |
| rs7181301 | 1 | 1.2855 | 0.4564 | 7.9330 | 0.0049 |
| rs913735 | 1 | −1.1026 | 0.3481 | 10.0342 | 0.0015 |
| ab_quar1 | 1 | 0.7188 | 0.2266 | 10.0573 | 0.0015 |
| stricture1 | 1 | 2.7013 | 0.4226 | 40.8556 | <.0001 |
| ip1 | 1 | 1.9157 | 0.5121 | 13.9936 | 0.0002 |

Hosmer and Lemeshow Goodness-of-Fit Test

| Chi-Square | DF | Pr > ChiSq | |
|---|---|---|---|
| 3.9729 | 8 | 0.8596 | AUC = 0.960 |

TABLE 14b
Odds Ratio Estimates

| | |
|---|---|
| Point | 95% Wald |

| Effect | Estimate | Confidence | Limits |
|---|---|---|---|
| rs6491869 | 0.823 | 1.205 | 64.638 |
| rs7575216 | 0.045 | 0.004 | 0.518 |
| rs10825455 | 0.355 | 0.188 | 0.669 |
| rs261804 | 2.312 | 1.387 | 3.853 |
| rs2993493 | 0.373 | 0.143 | 0.974 |
| rs1749969 | 0.358 | 0.164 | 0.782 |
| rs1325607 | 0.350 | 0.135 | 0.907 |
| rs1403146 | 0.441 | 0.250 | 0.777 |
| rs261827 | 0.360 | 0.194 | 0.668 |
| rs487675 | 0.376 | 0.217 | 0.651 |
| rs12386815 | 0.401 | 0.164 | 0.932 |
| rs2698174 | 0.200 | 0.141 | 0.955 |
| rs705308 | 10.337 | 3.508 | 30.461 |
| rs7181301 | 3.617 | 1.478 | 0.847 |
| rs913735 | 0.332 | 0.168 | 0.657 |
| ab_quar1 | 2.052 | 1.316 | 3.199 |
| stricture1 | 14.898 | 6.587 | 34.109 |
| ip1 | 6.792 | 2.489 | 18.930 |

### Example 17

### Model 4: Logistic Regression of Surgery with 30 SNPs Selected, Sex1, Age at Diagnosis 2, Sb1, Anca p1, and Antibody Sum

As demonstrated in Table 15, in the fourth model, 17 out of 30 SNPs, disease location, ANCA, and antibody sum were statistically significant.

TABLE 15a
Analysis of Maximum Likelihood Estimates

| | | | Standard | Wald | |
|---|---|---|---|---|---|
| Parameter | DF | Estimate | Error | Chi-Square | Pr > ChiSq |
| Intercept | 1 | 0.4807 | 1.9985 | 18.0074 | <.0001 |
| rs9742643 | 1 | 1.0930 | 0.3089 | 12.5188 | 0.0004 |
| rs10825455 | 1 | −1.0907 | 0.2890 | 14.2429 | 0.0002 |
| rs261804 | 1 | 0.6599 | 0.2991 | 4.8690 | 0.0273 |
| rs501691 | 1 | 0.6255 | 0.3241 | 3.7246 | 0.0536 |
| rs2993493 | 1 | −0.9194 | 0.4416 | 4.3349 | 0.0373 |
| rs1749969 | 1 | −0.9184 | 0.3430 | 7.1708 | 0.0074 |
| rs1325607 | 1 | −1.2065 | 0.4189 | 8.2937 | 0.0040 |
| rs1403146 | 1 | −1.0123 | 0.2577 | 15.4330 | <.0001 |
| rs261827 | 1 | −1.0659 | 0.2764 | 14.8709 | 0.0001 |
| rs487675 | 1 | −0.0561 | 0.2573 | 11.0698 | 0.0009 |
| rs12386815 | 1 | −1.2401 | 0.4158 | 8.8951 | 0.0029 |
| rs2698174 | 1 | −1.1881 | 0.3266 | 13.2361 | 0.0003 |
| rs705308 | 1 | 2.1105 | 0.4805 | 19.2958 | <.0001 |
| rs11845504 | 1 | −0.4644 | 0.2754 | 2.8436 | 0.0917 |
| rs898716 | 1 | −1.4547 | 0.4623 | 9.9016 | 0.0017 |
| rs7181301 | 1 | 1.3742 | 0.4276 | 10.3287 | 0.0013 |
| rs913735 | 1 | −0.7096 | 0.2998 | 5.6013 | 0.0179 |
| sb1 | 1 | 1.4676 | 0.4396 | 11.1446 | 0.0008 |
| anca_P1 | 1 | −1.0562 | 0.4430 | 5.6828 | 0.0171 |
| ab_sum | 1 | 0.5304 | 0.1458 | 13.2379 | 0.0003 |

Hosmer and Lemeshow Goodness-of-Fit Test

| Chi-Square | DF | Pr > ChiSq | |
|---|---|---|---|
| 4.8880 | 8 | 0.7695 | AUC = 0.940 |

TABLE 15b
Odds Ratio Estimates

| | |
|---|---|
| Point | 95% Wald |

| Effect | Estimate | Confidence | Limits |
|---|---|---|---|
| rs9742643 | 2.983 | 1.628 | 5.466 |
| rs10825455 | 0.336 | 0.191 | 0.592 |
| rs261804 | 1.935 | 1.077 | 3.477 |
| rs501691 | 1.869 | 0.990 | 3.528 |
| rs2993493 | 0.399 | 0.168 | 0.948 |
| rs1749969 | 0.399 | 0.204 | 0.782 |
| rs1325607 | 0.299 | 0.132 | 0.680 |
| rs1403146 | 0.363 | 0.219 | 0.602 |
| rs261827 | 0.344 | 0.200 | 0.592 |
| rs487675 | 0.425 | 0.257 | 0.703 |
| rs12386815 | 0.289 | 0.128 | 0.654 |
| rs2698174 | 0.305 | 0.161 | 0.578 |
| rs705308 | 0.253 | 3.218 | 21.165 |
| rs11845504 | 0.628 | 0.366 | 1.078 |
| rs898716 | 0.233 | 0.094 | 0.578 |
| rs7181301 | 3.952 | 1.709 | 0.136 |
| rs913735 | 0.492 | 0.273 | 0.885 |
| sb1 | 4.339 | 1.833 | 10.269 |
| anca_P1 | 0.348 | 0.146 | 0.829 |
| ab_sum | 1.700 | 1.277 | 2.262 |

### Example 18

### Model 5: Logistic Regression of Surgery with 30 SNPs Selected, Sex1, Age at Diagnosis, Sb1, Anca p1, Antibody Sum, Stricture1, and Ip1

As indicated in Table 16, in the fifth model, 15 out of 30 SNPs, antibody sum, internal penetrating, and stricture were statistically significant.

TABLE 16a
Analysis of Maximum Likelihood Estimates

| | | | Standard | Wald | |
|---|---|---|---|---|---|
| Parameter | DF | Estimate | Error | Chi-Square | Pr > ChiSq |
| Intercept | 1 | 5.6515 | 2.3696 | 5.6884 | 0.0171 |
| rs6491069 | 1 | 2.3223 | 0.9716 | 5.7134 | 0.0168 |
| rs7579216 | 1 | −2.9085 | 1.1932 | 5.3420 | 0.0148 |
| rs10825455 | 1 | −1.0239 | 0.3229 | 10.0561 | 0.0015 |
| rs261804 | 1 | 0.8842 | 0.2594 | 11.6139 | 0.0007 |
| rs2993493 | 1 | −0.8840 | 0.4757 | 3.4529 | 0.0631 |
| rs1749969 | 1 | −0.9685 | 0.3946 | 6.0235 | 0.0141 |
| rs1329607 | 1 | −1.0257 | 0.4795 | 4.5760 | 0.0324 |
| rs1403146 | 1 | −0.8829 | 0.2859 | 9.5328 | 0.0020 |
| rs261827 | 1 | −1.0102 | 0.3148 | 10.3004 | 0.0013 |
| rs487675 | 1 | −0.0331 | 0.2726 | 11.7189 | 0.0006 |
| rs12386815 | 1 | −0.9113 | 0.4469 | 4.1578 | 0.0414 |
| rs2698174 | 1 | −1.2875 | 0.3497 | 13.8742 | 0.0002 |
| rs705308 | 1 | 2.2974 | 0.5546 | 17.1582 | <.0001 |
| rs7181301 | 1 | 1.3132 | 0.4518 | 8.4487 | 0.0037 |
| rs913735 | 1 | −1.1052 | 0.3484 | 10.0611 | 0.0015 |
| ab_sum | 1 | 0.4456 | 0.1671 | 7.1145 | 0.0076 |
| stricture1 | 1 | 2.7412 | 0.4228 | 42.0421 | <.0001 |
| ip1 | 1 | 1.9216 | 0.5117 | 14.1165 | 0.0002 |

Hosmer and Lemeshow Goodness-of-Fit Test

| Chi-Square | DF | Pr > ChiSq | |
|---|---|---|---|
| 8.7486 | 8 | 0.3639 | AUC = 0.958 |

TABLE 16b
Odds Ratio Estimates

| | |
|---|---|
| Point | 95% Wald |

| Effect | Estimate | Confidence | Limits |
|---|---|---|---|
| rs6491069 | 10.199 | 1.519 | 68.477 |
| rs7975216 | 0.055 | 0.005 | 0.566 |
| rs10825455 | 0.359 | 0.191 | 0.676 |
| rs261804 | 2.421 | 1.456 | 4.026 |
| rs2993493 | 0.413 | 0.163 | 1.050 |
| rs1749969 | 0.389 | 0.175 | 0.823 |
| rs1325607 | 0.359 | 0.140 | 0.918 |
| rs1403146 | 0.414 | 0.236 | 0.724 |
| rs261827 | 0.364 | 0.196 | 0.675 |
| rs487675 | 0.393 | 0.231 | 0.671 |
| rs12386815 | 0.402 | 0.167 | 0.965 |
| rs2698174 | 0.275 | 0.140 | 0.543 |
| rs705308 | 9.948 | 3.355 | 29.501 |
| rs7181301 | 3.718 | 1.534 | 0.013 |
| rs913735 | 0.331 | 0.167 | 0.656 |
| ab_sum | 1.561 | 1.125 | 2.166 |
| stricture1 | 19.505 | 6.770 | 35.507 |
| ip1 | 6.639 | 2.508 | 18.644 |

### Example 19

### Survival Analysis

In order to examine the disease phenotypes (complication and surgery) and the time to reach the disease status, a survival analysis was performed with a Cox regression model. First, in order to select significant SNPs, genome-wide survival analyses were performed with a Cox regression model, in which each SNP was a predictor. Second, stepwise variable selection was applied to Cox regression models (3 models for complication and 5 models for surgery) using SNPs selected, gender, age, disease location, ANCA, and antibody sum/antibody quartile score as predictors. Third, the survival functions obtained by the Kaplan-Meier (KM) estimator among subgroups of patients were compared, which were subgrouped with 25% quartile and 75% quartile of the genetic risk score calculated from the selected model in the second step for each regression model (group1 if risk score≤25% quartile, group 2 if 25% quartile<risk score<75% quartile, and group3 if risk score≥75% quartile). Finally, for each subgroup, the survival functions were compared across the models.

### Example 20

### Survival Analysis for Complication

For complication, 50 SNPs with p-values less than 5×10⁻⁵ were selected throughout the genome-wide survival analyses. 3 Cox regression models were considered as follows; In model 1, the following variables were used: 50 SNPs, gender, age, and disease location. In model 2, the following variables were used: 50 SNPs, gender, age, disease location, ANCA, and antibody quartile score. In model 3, the following variables were used: 50 SNPs, gender, age, disease location, ANCA, and antibody sum. For each model, stepwise variable selection determined statistically significant predictors, as indicated in Table 17.

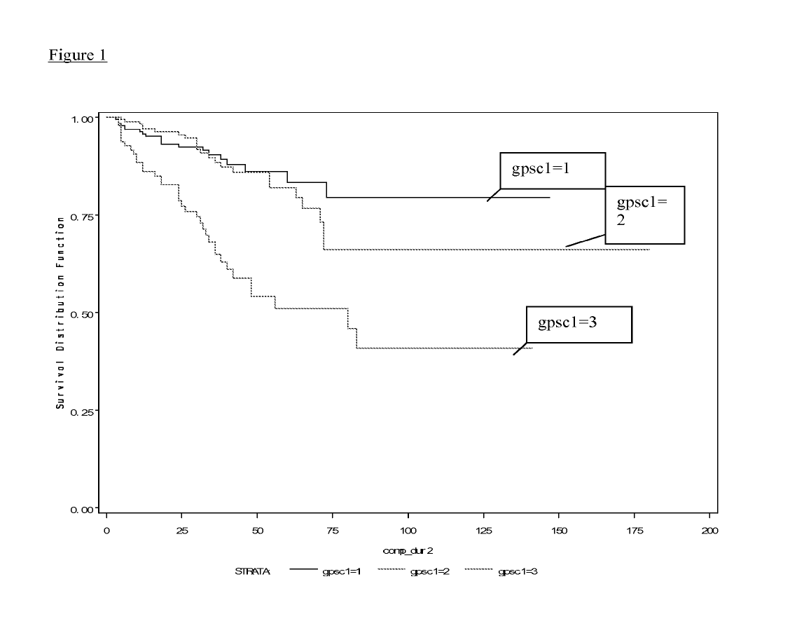

In the first model, 14 out of 50 SNPs, gender, and disease location were statistically significant. In the second model, 14 out of 50 SNPs, gender, disease location, and ANCA. In the third model, the results were the same as the model. For all 3 models, the survival functions obtained by the Kaplan-Meier (KM) estimator were significantly different among subgroups of patients (FIGS. 1,2). For all 3 subgroups, the survival functions across 3 models were statistically indistinguishable with a significance level of 0.05.

Tables 17-22 below indicate the results of the survival analysis for complication. As described herein, statistically significant predictors were identified for each model and used to determine a genetic risk score. The genetic risk score was then used to determine quartile subgroups. The column headings “minimum”, “median” and “maximum” in tables 17 and 23 refer to risk scores. The column headings “25% quartile” and “75% quartile” in tables 17 and 23 refer to boundaries for subgroups. The column heading “variable” in tables 17 and 23 refer to the model tested, ie. SC1 (model 1) or SC2 (model 2). The column heading “stratum” in each model refers to the range of risk scores within each group. The column heading “gp” in each model refers to the group number (ie. gpsc1 is group sc1 aka group 1). The column heading “N” in tables each model refers to the number of subjects used to calculate the results. The column heading “Failed” in tables 18-22 refers to the number of subjects experiencing complication. The column heading “Failed” in tables 23-30 refer to the number of subjects undergoing surgery. The column heading “Censored” in tables 18-22 indicates the number of subjects that did not experience complication as of a known date. The column heading “Censored” in tables 23-30 indicates the number of subjects that did not experience surgery as of a known date. The column headings “% Censored” and “Median” in tables 17-30 describe standard statistical manipulations of the data in each model.

TABLE 17
Survival for Complication

| Variable | Minimum | Median | Maximum | 25% Quartile | 75% Quartile |
|---|---|---|---|---|---|
| sc1 | 9 | 14 | 18 | 12 | 15 |
| sc2 | 9 | 15 | 19 | 13 | 16 |

### Example 21

### Survival for Complication Model 1: Summary of the Number of Censored and Uncensored Values and Test of Equality Over Strata

TABLE 18a
Model: SC1
Summary of the Number of Censored and Uncensored Values

| Stratum | gpsc1 | N | Failed | Censored | % Censored | Median |
|---|---|---|---|---|---|---|
| 1 (sc1 <= 12) | 1 | 190 | 20 | 170 | 89.47 | 32.0 |
| 2 (12 < sc1 < 15) | 2 | 176 | 23 | 153 | 86.93 | 31.5 |
| 3 (sc1 >= 15) | 3 | 97 | 36 | 61 | 62.89 | 31.0 |
| Total | | 463 | 79 | 384 | 82.94 | |

TABLE 18b
Test of Equality over Strata

| Test | Chi-Square | DF | Pr > Chi-Square |
|---|---|---|---|
| Log-Rank | 32.6525 | 2 | <.0001 |
| Wilcoxon | 31.1405 | 2 | <.0001 |
| −2Log(LR) | 26.9305 | 2 | <.0001 |

### Example 22

### Survival for Complication Model 2: Summary of the Number of Censored and Uncensored Values and Test of Equality Over Strata

TABLE 19a
Model: SC2
Summary of the Number of Censored and Uncensored Values

| Stratum | gpsc2 | N | Failed | Censored | % Censored | Median |
|---|---|---|---|---|---|---|
| 1 (sc2 <= 13) | 1 | 229 | 26 | 203 | 88.65 | 32.0 |
| 2 (13 < sc2 < 16) | 2 | 164 | 28 | 136 | 82.93 | 31.5 |
| 3 (sc2 >= 16) | 3 | 70 | 25 | 45 | 64.29 | 30.5 |
| Total | | 463 | 79 | 384 | 82.94 | |

TABLE 19b
Test of Equality over Strata

| Test | Chi-Square | DF | Pr > Chi-Square |
|---|---|---|---|
| Log-Rank | 22.3261 | 2 | <.0001 |
| Wilcoxon | 17.2221 | 2 | 0.0002 |
| −2Log(LR) | 18.6671 | 2 | <.0001 |

### Example 23

### Survival for Complication Stratum 1: Analysis Across Models

TABLE 20a
Across Models for Stratum 1
Summary of the Number of Censored and Uncensored Values

| Stratum | gp1 | N | Failed | Censored | % Censored |
|---|---|---|---|---|---|
| 1 | 1 | 190 | 20 | 170 | 89.47 |
| 2 | 2 | 229 | 26 | 203 | 88.65 |
| Total | | 419 | 46 | 373 | 89.02 |

TABLE 20b
Test of Equality over Strata

| Test | Chi-Square | DF | Pr > Chi-Square |
|---|---|---|---|
| Log-Rank | 0.0593 | 1 | 0.8075 |
| Wilcoxon | 0.0332 | 1 | 0.8555 |
| −2Log(LR) | 0.0492 | 1 | 0.8245 |

### Example 24

### Survival for Complication Stratum 2: Analysis Across Models

TABLE 21a
Across Models for Stratum 2
Summary of the Number of Censored and Uncensored Values

| Stratum | gp2 | N | Failed | Censored | % Censored |
|---|---|---|---|---|---|
| 1 | 1 | 176 | 23 | 153 | 86.93 |
| 2 | 2 | 164 | 28 | 136 | 82.93 |
| Total | | 340 | 51 | 289 | 85.00 |

TABLE 21b
Test of Equality over Strata

| Test | Chi-Square | DF | Pr > Chi-Square |
|---|---|---|---|
| Log-Rank | 0.8536 | 1 | 0.3555 |
| Wilcoxon | 1.2619 | 1 | 0.2613 |
| −2Log(LR) | 0.9108 | 1 | 0.3399 |

### Example 25

### Survival for Complication Stratum 3: Analysis Across Models

TABLE 22a
Across Models for Stratum 3
Summary of the Number of Censored and Uncensored Values

| Stratum | gp3 | N | Failed | Censored | % Censored |
|---|---|---|---|---|---|
| 1 | 1 | 97 | 36 | 61 | 62.89 |
| 2 | 2 | 70 | 25 | 45 | 64.29 |
| Total | | 167 | 61 | 106 | 63.47 |

TABLE 22b
Test of Equality over Strata

| Test | Chi-Square | DF | Pr > Chi-Square |
|---|---|---|---|
| Log-Rank | 0.0023 | 1 | 0.9621 |
| Wilcoxon | 0.0271 | 1 | 0.8693 |
| −2Log(LR) | 0.0008 | 1 | 0.9779 |

### Example 26

### Survival Analysis for Surgery

For surgery, 75 SNPs were selected throughout the genome-wide survival analyses with the p-value (10⁻⁵). Similarly to the complication, 5 Cox regression models were considered. In model 1, the following variables were used: 75 SNPs, gender, age, and disease location. In model 2, the following variables were used: 75 SNPs, gender, age, disease location, ANCA, and antibody quartile score. In model 3, the following variables were used: 75 SNPs, gender, age, disease location, ANCA, antibody quartile score, internal penetrating, and stricture. In model 4, the following variables were used: 75 SNPs, gender, age, disease location, ANCA, and antibody quartile score. In model 5, the following variables were used: 75 SNPs, gender, age, disease location, ANCA, antibody quartile score, internal penetrating, and stricture. For each model, stepwise variable selection. In the first model, 12 out of 75 SNPs, age, and disease location were statistically significant. In the second model: 11 out of 75 SNPs, disease location, and antibody quartile were statistically significant. In the third model, 7 out of 75 SNPs, internal penetrating, and stricture, were statistically significant. In the fourth model, 15 out of 75 SNPs, disease location, and antibody sum were statistically significant. For all 5 models, the survival functions obtained by the Kaplan-Meier (KM) estimator indicated significant differences among subgroups of patients. For all 3 subgroups, the survival functions across the 5 models were statistically indistinguishable, with a significance level of 0.05.

TABLE 23
Survival for Surgery

| | | | | 25% | 75% |
|---|---|---|---|---|---|
| Variable | Minimum | Median | Maximum | Quartile | Quartile |
| ss1 | 2 | 5 | 11 | 4 | 6 |
| ss2 | 3 | 6 | 13 | 5 | 7.5 |
| ss3 | 1 | 3 | 8 | 2 | 4 |
| ss4 | 7 | 11 | 20 | 10 | 12 |

### Example 27

### Survival for Surgery Model 1: Summary of the Number of Censored and Uncensored Values and Test of Equality Over Strata

TABLE 24a
SS1 Model
Summary of the Number of Censored and Uncensored Values

| Stratum | gpss1 | N | Failed | Censored | % Censored | Median |
|---|---|---|---|---|---|---|
| 1 (ss1 >= 4) | 1 | 430 | 33 | 397 | 92.33 | 33 |
| 2 (4 < ss1 < 6) | 2 | 53 | 20 | 33 | 62.26 | 34 |
| 3 (ss1 >= 6) | 3 | 53 | 33 | 20 | 37.74 | 26 |
| Total | | 536 | 86 | 450 | 83.96 | |

TABLE 24b
Test of Equality over Strata

| Test | Chi-Square | DF | Pr > Chi-Square |
|---|---|---|---|
| Log-Rank | 181.4000 | 2 | <.0001 |
| Wilcoxon | 130.1560 | 2 | <.0001 |
| −2Log(LR) | 99.0692 | 2 | <.0001 |

### Example 28

### Survival for Surgery Model 2: Summary of the Number of Censored and Uncensored Values and Test of Equality Over Strata

TABLE 25a
SS2 Model
Summary of the Number of Censored and Uncensored Values

| Stratum | gpss2 | N | Failed | Censored | % Censored | Median |
|---|---|---|---|---|---|---|
| 1 (ss2 >= 5) | 1 | 423 | 29 | 394 | 93.14 | 34 |
| 2 (5 < ss2 < 7.5) | 2 | 83 | 37 | 46 | 55.42 | 30 |
| 3 (ss2 >= 7.5) | 3 | 30 | 20 | 10 | 33.33 | 24 |
| Total | | 536 | 86 | 450 | 83.96 | |

TABLE 25b
Test of Equality over Strata

| Test | Chi-Square | DF | Pr > Chi-Square |
|---|---|---|---|
| Log-Rank | 198.0272 | 2 | <.0001 |
| Wilcoxon | 134.8483 | 2 | <.0001 |
| −2Log(LR) | 111.3678 | 2 | <.0001 |

### Example 29

### Survival for Surgery Model 3: Summary of the Number of Censored and Uncensored Values and Test of Equality Over Strata

TABLE 26a
SS3 Model
Summary of the Number of Censored and Uncensored Values

| Stratum | gpss2 | N | Failed | Censored | % Censored | Median |
|---|---|---|---|---|---|---|
| 1 (ss3 >= 2) | 1 | 346 | 22 | 324 | 93.64 | 35 |
| 2 (2 < ss3 < 4) | 2 | 105 | 23 | 82 | 78.10 | 30 |
| 3 (ss3 >= 4) | 3 | 85 | 41 | 44 | 51.76 | 29 |
| Total | | 536 | 86 | 450 | 83.96 | |

TABLE 26b
Test of Equality over Strata

| Test | Chi-Square | DF | Pr > Chi-Square |
|---|---|---|---|
| Log-Rank | 120.8535 | 2 | <.0001 |
| Wilcoxon | 97.2703 | 2 | <.0001 |
| −2Log(LR) | 83.8218 | 2 | <.0001 |

### Example 30

### Survival for Surgery Model 4: Summary of the Number of Censored and Uncensored Values and Test of Equality Over Strata

TABLE 27a
SS4 Model
Summary of the Number of Censored and Uncensored Values

| Stratum | gpss2 | N | Failed | Censored | % Censored | Median |
|---|---|---|---|---|---|---|
| 1 (ss3 >= 10) | 1 | 456 | 39 | 417 | 91.45 | 33 |
| 2 (10 < ss3 < 12) | 2 | 38 | 21 | 17 | 44.74 | 32 |
| 3 (ss3 >= 12) | 3 | 42 | 26 | 16 | 38.10 | 24 |
| Total | | 536 | 86 | 450 | 83.96 | |

TABLE 27b
Test of Equality over Strata

| Test | Chi-Square | DF | Pr > Chi-Square |
|---|---|---|---|
| Log-Rank | 171.1712 | 2 | <.0001 |
| Wilcoxon | 138.5943 | 2 | <.0001 |
| −2Log(LR) | 93.0443 | 2 | <.0001 |

### Example 31

### Survival for Surgery Stratum 1: Analysis Across Models

TABLE 28a
Across Models for Stratum 1
Summary of the Number of Censored and Uncensored Values

| Stratum | gp1 | N | Failed | Censored | % Censored |
|---|---|---|---|---|---|
| 1 | 1 | 430 | 33 | 397 | 92.33 |
| 2 | 2 | 423 | 29 | 394 | 93.14 |
| 3 | 3 | 346 | 22 | 324 | 93.64 |
| 4 | 4 | 456 | 39 | 417 | 91.45 |
| Total | | 1655 | 123 | 1532 | 92.57 |

TABLE 28b
Test of Equality over Strata

| Test | Chi-Square | DF | Pr > Chi-Square |
|---|---|---|---|
| Log-Rank | 2.1519 | 3 | 0.5415 |
| Wilcoxon | 2.2926 | 3 | 0.5139 |
| −2Log(LR) | 1.9439 | 3 | 0.5841 |

### Example 32

### Survival for Surgery Stratum 2: Analysis Across Models

TABLE 29a
Across Models for Stratum 2
Summary of the Number of Censored and Uncensored Values

| Stratum | gp2 | N | Failed | Censored | % Censored |
|---|---|---|---|---|---|
| 1 | 1 | 53 | 20 | 33 | 62.26 |
| 2 | 2 | 83 | 37 | 46 | 55.42 |
| 3 | 3 | 143 | 44 | 99 | 69.23 |
| 4 | 4 | 143 | 44 | 99 | 69.23 |
| Total | | 422 | 145 | 277 | 65.64 |

TABLE 29b
Test of Equality over Strata

| Test | Chi-Square | DF | Pr > Chi-Square |
|---|---|---|---|
| Log-Rank | 7.7332 | 3 | 0.0519 |
| Wilcoxon | 2.9542 | 3 | 0.3987 |
| −2Log(LR) | 5.7950 | 3 | 0.1220 |

### Example 33

### Survival for Surgery Stratum 3: Analysis Across Models

TABLE 30a
Across Models for Stratum 3
Summary of the Number of Censored and Uncensored Values

| Stratum | gp3 | N | Failed | Censored | % Censored |
|---|---|---|---|---|---|
| 1 | 1 | 53 | 33 | 20 | 37.74 |
| 2 | 2 | 30 | 20 | 10 | 33.33 |
| 3 | 3 | 85 | 41 | 44 | 51.76 |
| 4 | 4 | 42 | 26 | 16 | 38.10 |
| Total | | 210 | 120 | 90 | 42.86 |

TABLE 30b
Test of Equality over Strata

| Test | Chi-Square | DF | Pr > Chi-Square |
|---|---|---|---|
| Log-Rank | 7.0961 | 3 | 0.0689 |
| Wilcoxon | 4.2355 | 3 | 0.2371 |
| −2Log(LR) | 5.5109 | 3 | 0.1380 |

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

1. A method of prognosing Crohn's disease in an individual, comprising:
obtaining a sample from the individual;
assaying the sample for the presence or absence of one or more genetic risk variants; and
prognosing an aggressive form of Crohn's disease based on the presence of one or more genetic risk variants,
wherein the one or more genetic risk variants are selected from the genetic loci of 8q24, 16p11, Bromodomain and WD repeat domain containing 1 (BRWD1) and/or Tumor necrosis factor superfamily member 15 (TNFSF15).

2. The method of claim 1, wherein the presence of each genetic risk variant has an additive effect on rapidity of Crohn's disease progression from a relatively less severe case of Crohn's disease to a relatively more severe case of Crohn's disease.

3. The method of claim 1, wherein the one or more genetic risk variants comprise SEQ. ID. NO.: 1, SEQ. ID. NO.: 2, SEQ. ID. NO.: 3, SEQ. ID. NO.: 4, SEQ. ID. NO.: 5 and/or SEQ. ID. NO.: 6.

4. The method of claim 1, wherein the aggressive form of Crohn's disease is characterized by one or more phenotypes associated with complications.

5. The method of claim 1, wherein the aggressive form of Crohn's disease is characterized by one or more phenotypes associated with conditions requiring surgery.

6. The method of claim 1, wherein the aggressive form of Crohn's Disease is characterized by a rapid progression from a relatively less severe case of Crohn's disease to a relatively more severe case of Crohn's disease.

7. The method of claim 1, wherein the individual has previously been diagnosed with inflammatory bowel disease (IBD).

8. The method of claim 1, wherein the individual is a child 17 years old or younger.

9. The method of claim 1, wherein the aggressive form of Crohn's disease comprises internal penetrating and/or stricture.

10. The method of claim 1, wherein the aggressive form of Crohn's disease comprises a high expression of anti-neutrophil cytoplasmic antibody (ANCA) relative to levels found in a healthy individual.

11. The method of claim 1, wherein the presence of one or more genetic risk variants is determined from an expression product thereof.

12. A method of prognosing Crohn's disease in an individual, comprising:
obtaining a sample from the individual;
assaying the sample for the presence or absence of one or more genetic risk variants; and
prognosing a form of Crohn's disease associated with a complication based on the presence of one or more genetic risk variants,
wherein the one or more genetic risk variants is selected from the group consisting of SEQ. ID. NO.: 7, SEQ. ID. NO.: 8, SEQ. ID. NO.: 9, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, SEQ. ID. NO.: 18, SEQ. ID. NO.: 19, SEQ. ID. NO.: 20, SEQ. ID. NO.: 21, and/or SEQ. ID. NO.: 22.

13. The method of claim 12, wherein the complication comprises internal penetrating and/or stricturing disease.

14. A method of prognosing Crohn's disease in an individual, comprising:
obtaining a sample from the individual;
assaying the sample for the presence or absence of one or more genetic risk variants; and
prognosing a form of Crohn's disease associated with one or more conditions that require a treatment by surgery;
wherein the one or more genetic risk variants is selected from the group consisting of SEQ. ID. NO.: 23, SEQ. ID. NO.: 24, SEQ. ID. NO.: 25, SEQ. ID. NO.: 26, SEQ. ID. NO.: 27, SEQ. ID. NO.: 28, SEQ. ID. NO.: 29, SEQ. ID. NO.: 30, SEQ. ID. NO.: 31, SEQ. ID. NO.: 32, SEQ. ID. NO.: 33, SEQ. ID. NO.: 34, SEQ. ID. NO.: 35, SEQ. ID. NO.: 36, SEQ. ID. NO.: 37, SEQ. ID. NO.: 38, SEQ. ID. NO.: 39, SEQ. ID. NO.: 40, SEQ. ID. NO.: 41, SEQ. ID. NO.: 42, SEQ. ID. NO.: 43, SEQ. ID. NO.: 44, SEQ. ID. NO.: 45, SEQ. ID. NO.: 46, SEQ. ID. NO.: 47, SEQ. ID. NO.: 48, SEQ. ID. NO.: 49, SEQ. ID. NO.: 50, SEQ. ID. NO.: 51, and/or SEQ. ID. NO.: 52.

15. The method of claim 14, wherein the treatment by surgery comprises small-bowel resection, colectomy and/or colonic resection.

16. A method of treating Crohn's disease in an individual, comprising:
prognosing an aggressive form of Crohn's disease in the individual based on the presence of one or more genetic risk variants; and
treating the individual,
wherein the one or more genetic risk variants are selected from the genetic loci of 8q24, 16p11, Bromodomain and WD repeat domain containing 1 (BRWD1) and/or Tumor necrosis factor superfamily member 15 (TNFSF15).

17. The method of claim 16, wherein treating the individual comprises exposing the individual to a treatment that ameliorates the symptoms of Crohn's disease on the basis that the subject tests positive for one or more genetic risk variants.

18. The method of claim 16, wherein treating the individual comprises administering a surgical procedure associated with treating an aggressive form of Crohn's disease.

19. The method of claim 16, wherein treating the individual comprises performing on the individual a small-bowel resection, colectomy and/or colonic resection.

20. The method of claim 16, wherein the presence of each genetic risk variant has an additive effect on rapidity of Crohn's disease progression from a relatively less severe case of Crohn's disease to a relatively more severe case of Crohn's disease.

21. The method of claim 16, wherein the one or more genetic risk variants comprise SEQ. ID. NO.: 1, SEQ. ID. NO.: 2, SEQ. ID. NO.: 3, SEQ. ID. NO.: 4, SEQ. ID. NO.: 5 and/or SEQ. ID. NO.: 6.

22. The method of claim 16, wherein the one or more genetic risk variants comprise SEQ. ID. NO.: 7, SEQ. ID. NO.: 8, SEQ. ID. NO.: 9, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, SEQ. ID. NO.: 18, SEQ. ID. NO.: 19, SEQ. ID. NO.: 20, SEQ. ID. NO.: 21, and/or SEQ. ID. NO.: 22.

23. The method of claim 16, wherein the one or more genetic risk variants comprise SEQ. ID. NO.: 23, SEQ. ID. NO.: 24, SEQ. ID. NO.: 25, SEQ. ID. NO.: 26, SEQ. ID. NO.: 27, SEQ. ID. NO.: 28, SEQ. ID. NO.: 29, SEQ. ID. NO.: 30, SEQ. ID. NO.: 31, SEQ. ID. NO.: 32, SEQ. ID. NO.: 33, SEQ. ID. NO.: 34, SEQ. ID. NO.: 35, SEQ. ID. NO.: 36, SEQ. ID. NO.: 37, SEQ. ID. NO.: 38, SEQ. ID. NO.: 39, SEQ. ID. NO.: 40, SEQ. ID. NO.: 41, SEQ. ID. NO.: 42, SEQ. ID. NO.: 43, SEQ. ID. NO.: 44, SEQ. ID. NO.: 45, SEQ. ID. NO.: 46, SEQ. ID. NO.: 47, SEQ. ID. NO.: 48, SEQ. ID. NO.: 49, SEQ. ID. NO.: 50, SEQ. ID. NO.: 51, and/or SEQ. ID. NO.: 52.

24. The method of claim 16, wherein the individual is a child 17 years old or younger.

25. A method of diagnosing susceptibility to Crohn's disease in an individual, comprising:
obtaining a sample from the individual;
assaying the sample for the presence or absence of one or more genetic risk variants; and
diagnosing susceptibility to Crohn's disease in the individual based on the presence of one or more genetic risk variants,
wherein the one or more genetic risk variants are located at the genetic loci of 8q24, 16p11, and/or Bromodomain and WD repeat domain containing 1 (BRWD1).

26. The method of claim 25, wherein the one or more genetic risk variants comprise SEQ. ID. NO.: 1, SEQ. ID. NO.: 2, SEQ. ID. NO.: 3, SEQ. ID. NO.: 4, SEQ. ID. NO.: 5 and/or SEQ. ID. NO.: 6.

27. The method of claim 25, wherein the one or more genetic risk variants comprise SEQ. ID. NO.: 7, SEQ. ID. NO.: 8, SEQ. ID. NO.: 9, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, SEQ. ID. NO.: 18, SEQ. ID. NO.: 19, SEQ. ID. NO.: 20, SEQ. ID. NO.: 21, and/or SEQ. ID. NO.: 22.

28. The method of claim 25, wherein the one or more genetic risk variants comprise SEQ. ID. NO.: 23, SEQ. ID. NO.: 24, SEQ. ID. NO.: 25, SEQ. ID. NO.: 26, SEQ. ID. NO.: 27, SEQ. ID. NO.: 28, SEQ. ID. NO.: 29, SEQ. ID. NO.: 30, SEQ. ID. NO.: 31, SEQ. ID. NO.: 32, SEQ. ID. NO.: 33, SEQ. ID. NO.: 34, SEQ. ID. NO.: 35, SEQ. ID. NO.: 36, SEQ. ID. NO.: 37, SEQ. ID. NO.: 38, SEQ. ID. NO.: 39, SEQ. ID. NO.: 40, SEQ. ID. NO.: 41, SEQ. ID. NO.: 42, SEQ. ID. NO.: 43, SEQ. ID. NO.: 44, SEQ. ID. NO.: 45, SEQ. ID. NO.: 46, SEQ. ID. NO.: 47, SEQ. ID. NO.: 48, SEQ. ID. NO.: 49, SEQ. ID. NO.: 50, SEQ. ID. NO.: 51, and/or SEQ. ID. NO.: 52.

29. The method of claim 25, wherein the individual is a child 17 years old or younger.


SEQUENCE LISTING (52 sequences)

SEQ ID NO: 1 - DNA, 879 bp, Homo sapiens
ggaggtgacagtgagctgaaccccagaagatgagaagaagccagcagtgcaaagagctgggggtagaattttccagaaaatgttcaccaatgcaaagtccacaaggtagtgagaaaggctggctggcttgctcaaagattaagaaggggctggactggctggcgtatgcagggtgagggaaaggggtgaggcctgagatgcctccaggtgccaggtcatgctggacatggcaggagttaggacttgatttggagggagattttaaacacattgtaggatccttcccttcctggagccttgggtcccaaatgcctgggggacaagggtaaccaacacaactcttgctcacaggcacaggaaggaaccgcagagtctgtccagatcaatgcccttccactttgtagatgggtaacagcccagagatgggaagggacgtgcacaagatgggaatgggcgtgcccatggttgcaccgtgtggtgtggcagagcaggaactggaayacaggcggctggaagtgaaagtggagctcaggctttttagcagttactatgtgtgatttccttttcatcatcacatcaaccccatttttttttttcagatgagaaagggaaagtgacctcctaagattccacagcgagaggtgctaggggagccaggctccaaattaaggtcagcacccaaactctttccactgggctgccttggatttacatagatatccctacagtcccaagctgctaagtccagacctagcacttactgaaagctgctggcagtttctctgggaaataccatgagttacaagcagatgaaacaaatgagccagttgccctcacgcatcagtccattaagaaacaaatggaaatacattttttaaa

SEQ ID NO: 2 - DNA, 701 bp, Homo sapiens
aaatgtttggaaggtagagaaaagtaaccaatagacccactattgtaactggattgtttttgtgaattgttatagtttttgaaaaaataattgttctgctataacttttatattctgctattctcacatcatatttatcattcctttaaaattttctagttttagagtggagactatgttcttttttttttttttttttttgagacagggtcttgctatgttgtccagactggagtgcagtggcatgatcacagctcactgtagcctccacctcccaggctcaagcaatcctcccacctcagcctcctgagtaactatgactacagacacaccaccacacctggctaatttttgtattttttatagagatgaggttttgccctgttgcccaggctggtctcttaactcctggactcaagcaatctgcctgcctcggcctcccaaagtgctgggattacaggcttgagccacggcacctgtctatgttctcttttcactgtmggatcagagggtcatattgatggatgatagtgatggttactttgcattttggaatggtaaacttaaacatgaactgttatacagtactctactgaatcttttaaaattcttttggtgtttttgcaaataggtatgagcattctccagggaaaggatgacatatttttggacctgaaacagaaattctggaacacctatat

SEQ ID NO: 3 - DNA, 401 bp, Homo sapiens
atagctgtaacactgtgtgcaggtatctggggtttctgtcgtgaccacgtggcaggagctgctgccactgctgtctgatgctcgccccacagtggaaggagatgctaaattccgttacgcattagaggtcagtgaaaaggaagatgcagtttgttcccgtccaggcacaaggactcttgaatttgtccatagttaagaacrgctcatccaggagcagagcgagaggccgggctgcgcgtcctcatctcctctcccagccttcgcatcctcctggctgcctcgcgtttcctccacgggcctggctgaacgcacacacaggcctgggggagactgcagagacacatcttcagccacatcttctgtaaaacagtcactatgggatgacggtgactggacagtgg

SEQ ID NO: 4 - DNA, 801 bp, Homo sapiens
gattgtagccctatgttatctgttgtctaatgtccaattttatagttgttataataagcaggcaagtcctgtagccattgctctgtcttggctagaactgggagttccaatgttttcttcatcttttttttttttctcagaacttagagtagttcctagaactaggtgcttggggttagttgaatgaatacacaataattttgttatgttgacctggataagggcacatggcaaagcaaaatgtagtagacccctggtctctggtctcttggtgtgggctattctatgctgcatcacacagggcttcattttcttctctgtggttctgaattggggatcacagttcactcaacatgtacctttgtggataaaagccttaagttccccatgaatgacttttyccccctcctttataaaattgacacccatgcttgtgatgaaaccacatttaaccttttgggattcactttgcactttgggggactgtgggggaagcacacagaaatgtctggaattttttcagtactatcttcttagcttccttcttcatttaacaaagccacagaacatggcttagatcatttgtctgataaaatgcggagctactttcctggtttcttgaataattctgttgattagccaggtcctcttggggcaactgtttattggcctacattctttcaagttgatgctgaaatattgatttttatttaactcactttcttaagtgacttaaatataaattaagtggctgcagggggaagtggggtacagtttaagtggctgcagagggaagtgggg

SEQ ID NO: 5 - DNA, 501 bp, Homo sapiens
agttgtttctgtaggcctgtgctgaatagtgctgcagggaaaaaagacaaatttgagggctgggtactttaatgttaaaatatttaagtttaaatttttgtgagacttttgctaagtcctgtggctgatgttgagaaaacaatgcacttggttccaagcatgttgaggatgtagtgttgtgaaaagtttgggaagggtaagagaaatccagttctatttaagagaaatccagttctatttttgccttcacytttcttgaaactgacccatgggtgtggggaatggggtgtttgtagtttgaactagccgtggatgctgtgcaccggaaagcatctgagccagcaggtggccctcgtcgggaggacattgtggacaccatggtgtttaagccaagtgatgtcatgcttgttcacttccgaaatgttgacttcaactatgctactaaaggtattgtcctaggctgttacctcagacctgctctgtgtgcatagaggacagagg

SEQ ID NO: 6 - DNA, 1041 bp, Homo sapiens
cttacttcacacagcagtgcaaagctctaatcagagcttttcaaacctccaaactcacacaaacacatactcaaattagtctcaattgtattcgattctcaaacctaggcttcaaggttcaagtctccttttttcatcaacggagctctctctctccccctgaagtgtgtgccaaacactgattttcctcctgtaaagtgaaggcttggaccagattaggtctttcctaaaaatcaatctgtgagcctgtactggtccacagagaagtttcctctgatttgcagacagttatcttgttccttatacagtcttaacttcacggagttcaaccatgaaagactgtgtttttctctacttacttcacacagcagtgctttgtagaactgcccacagtttctcatagatagcagagttctcaaatccttttcctcaatttgtgtaagtccatttgctttaaaacttcaatcttatatctgggttaaagctttttccttcatctcaaatgggctccaagtgacatygtagccctaccacatgatagtttttcaggccactccagtgctctgctcccatcatcttccgtgttcaccccactcccctgctgccatcatgctccagtgttcctacttaagtagacacaggaggcagattagcaaggacgcctcataagtgcatgtctagctaggcaatgggatgttagcctctcccagtcaagcaggtccctctcatgtttaccaatgattttcttgatctgtattggttggtttgggatgtgaggaagacccctctcttcccacatgggtgctctctttagaatacaggctacttaccagcccctttgtcctctgctgctttggacccccacccatccacagcatcaattcagggcaataaaaatatgtcccactcaccatcctattattttttcttgtgtgtcagtatgaagttgcctaatattctttgtttgaaagtattgtccttcatttcaagattatattctactatctttttaaaaaaaaattgttctgcaggcatccttgt

SEQ ID NO: 7 - DNA, 628 bp, Homo sapiens
gaggctcccagagaccctgcccttggctggacaccctgaagttctcatgggggactgagctgcagatgcctgcagggactgggtacctgggcatggaggaggcaaatagtcagggcccacaggcccaggtcatcctacccccatccacagccatggagactgatttaatagtaatattaaacataatgaataataataaataatgctatttattgtaaaaccacttagcaaagtcgaagtagmccctaagattagtgtcactttcattattgttatttttacatgtcagttgctttacatggattatctcatttaattctcgtaacgaccctatgagatcagtcttacaatttttttttcttggcttttgacagcccaagatgcagaggctcagatgggacaaacaggttccttgtgcagggtcatgtgggtaccaagtgttggaggcaggatgagaagcaagcaatccacttccactgaggccccacctcccactgttgttagctcagtgccaatagctgccttttactccacgtcatggacagtctagcccatggtacgccgctaactggcctctccatcttgaccaaacctccacaaggcctcaggtttgcagggtcctgtagac

SEQ ID NO: 8 - DNA, 3112 bp, Homo sapiens
acaggcgcccgccactacgcccggctaattttttgtatttttagtagagacggggtttcaccgttttagccgggatggtctcgatctcctgacctcgtgatccgcccgcctcggcctcccaaagtgctgggattacaggcatgagccaccacgcccggcgaacattgacaaacttttagtgagattgactgagaaaaaaggaaaaactcaaaatgctaaaatcaggaataaaagagcacatcactaccaactttacagaaataaaaagaatcagaaggaggagcttatgaataattgtatatcagcaaattaggttacctagatgaaatggacaaattcctagaaacacacaaactttcaatactggttcaagaagaaacagaaaatctgaatatatctatataattagtaaagaaattgaattagtaatcaaaatgcttcacaaaaagaaatgcttcattggtcaattctatgaaatgtctaaagcagaattaacatcaatccttcataaactcttccaaaatatagaagaggagaaagcacttctcaactcattcactaaggccattatgaccctgatacaaaaaccagaaaaagacatcctagatgtcctaacagaatgctatcaaactgaatccagcaacatatgaaaaggagtatgtaccattgccaagtgaaatgtattgtaggaatacaaaccttgatctaatacgtgaaaatcactatggtaatagtaatagaatcaaggacaaaaaccatagacacagaaaacccacatggcaaaatttaacagcttttaatgatgaaaacattcaacaaactagaaatagaaggtgacttcttcagtccgatagggttttgtatgaaaaacccacagctaacatcataaatgatgaaagactgagtactttaagattagtaacaagacaaagacgtcctgtctctctactccttttcaaaattgtactggaggttccagccagggcgattaggcaggaaaaaaaattaaaagatatccacattagaaagaataaagtaaaattatctttattcacaaataatgtgatctcatacacacacaaaaaaatcctaggaaatctactagaaaaccattagagctaacatacacgttcaataacatttcaggatatgagattaacatacaaaaaacaattatatttctatacattagcaatgaacaatccaaaagtgaaattaagaaaacaattcaattcacaaaaacatcaggaagaataaaatactgaggaataaatttaacaaaataagtgcaagattttgtacactgaaaatgacaaaaaatttaaaagaaatgaaaaaacacataaacaggaacacattctatgtttatgggcaggacaacttaatattgataagatgacaatactcctcaaattgatttacagattcagtgcaattcctgtcaaaatcccagctgccttttttaaataaactgacaaactgatcctaaaattcatatgaaaatgtaaggaactcaaaatagccgaaactacctttaaaggcaacaaaaaagttgaggactcacacttcttgttcacaaaactatggtaatgaagacattatgctatggcataagaatagtcatatagatcaatggaattgaaatatatccagaaataaattcctacactgtggtcaaatgacttatgtcaaagatgccaagacaattcactgggggcaaaacttttttttcaacaagtgttacaaggacaactgaatatccacatgcaaagttgggtccctgccttaaaccatatgcaaaaaaaattttttttaatttcaaagtggatcaaagacttacatctaagagaaaaagtataaaaattttcagagaaagcataattaccttggattagataatggttgcttagctatgacaccaaaagcacaagcaagtaaagaaaaaatgaattggacttaaccaaaattttaaaatttggggacttcaaatggttccatcragaaagtttaaaaaaactcacaaaatgagagatatttgcaagtcagatatttgataaggatcttacacacaggatatatcaagaactatcatatttcaacaataaaaagacaatctaattttaagatgggcaaaagatttgaaaagacattcctccaaagaagagaggcaaatgctaataatacattaaaatatgcttaacatcattagtcactagaaaaatctaaataaaaagtttacaccaatttgggtggttagaatcaaaaagtcagataataacaagagttggtgacgatgtgaagaaattggaaatcttgtacattgttaatggtaatgtaaaatggtgtagccactttggaaagcaatttggcagttcctcaaaaagacaagcacagagttaccgtatgacccagcaattccccttctaggtagatacccaagagaatttaaaacatacccacacaaaaacctgtacaaaatgtttatagcagcattattcagaatagcaaatgtggaaacaacccaaatgctcaactgatgaaagaacagaaaaaaatgtgtcatatccatacacgttatggcataaaaggcagttaagcactgatacatgctatgacattttattggtcattaaaggcagtgaagaactgatccatgctatagcatggatgaaccctgagcacactatgctgagtggaagaagtcagacactgaggccacatattgtatgactacatgcatgtgaaatgtgtggaagaggcaaatccatgcaagagaaaatagattcgtgattcccagaggctgagagaagaaggaataggctgtgattgttagtgaggatgaggtttcttttaaaggtgataaaatgttctggaattagatagtggtgatgattgtacaatatgttacgaatatactttacactttaaaagggtgaatgttatgctatgtgagtcatacctcagtgaaattgtcataaatacacacacaccacacacacacacacacacacacactctgacggcagcagcagaggaaagctgctgttgtccagccagcctcacacagcccatcagaactgtagtggctctcagtgctcagggctgatggaga

SEQ ID NO: 9 - DNA, 601 bp, Homo sapiens
tttaggtaattattatatacttagtaattaagtaattattattaccattaaaagggatatctttggttgtgacagaacttgtgggcttagataaccaacaagcctagaaccaggggtagctttaagtaaggcttgatccagcagctgttaccaagaacctattttctttcagtctcttggctctgtctcctatgatgtgtcacaaggtggctgccagaagctcctggcaatacttgcttttctcctttgtaattagcaagaagtagttggtttcccagttagctcaaagaaaagactcagrattgagacccattcactctggttggcctgatttgggacctatacctttttctgaccaattgctgtagcctgaaggatgaaatattctgatttggctgggtgcagcggcttatgcctgtaatcccaggcactttgagaggccgagacagctggattgcctgagctcaggagttcaagaccagcctgggcaacatggggaaaccccatctctactaaaaatacaaaaaatggccggctgtggtggcatgcacctgtgatcccagctactcaggaggctgaggtgggaggatcacttgagcct

SEQ ID NO: 10 - DNA, 601 bp, Homo sapiens
tttaacaagaacccaacacctcaaacctagcaaaaaagcatagctcaggtcaatcacataaaacacagcttcacaaaatctgtcccataacaaagctagaaatacaatggtgtaataaatttggtatacctaagagcctgagaaaaggtacagctgcttaagaataagttatagaaaatctttgttaaatactctagggaaggattcaggcatcttcctacttatctaactttttagtgtattctaaacttggccaggaaggagaggtgtgatcttggcttggtgattcctgtagatctcyggactttggctgcctcacctttacctgaaactgttgaatgggatgctagattccctccagctctgacattccatgactaaagaaagtcaaggtttaacactgaccttgacatcctggggcaatggagagttttagtgtatgcaattgtcatgcctgaaaaaatgtcaaaacatggttagggttcctctccttgttcaggagaataaaatagtatattttaaatatgctagtataaatcttataaataaaatagtataaatctcccttctgttaagagaatgtagaaataaaataaataaa

SEQ ID NO: 11 - DNA, 601 bp, Homo sapiens
tctgcagctacttggctgtgggttcccagagagccctgaggcaacctttataaaaggcctaggtttgacttctctttggtcacttcagctttagagcaccctaagaaggctcagagaaccccaaatttcttcccagtaaagaggtcatggaataaatctggaggaactcaagaggggcgcctgcttttgaacagtcattagccaaacatgggtagattcctattaatcatgtcaagagggtacttgggaatcaagttcatgtggaattgggagattaaaagggttaggagtccttggagcygtaagacccctgaagttccaactatgctacttgctttctgtgtaatcttggataagaaatcacttgacctttctgagccttgatttccttacctgtaaaatgggaatgctaatcattgtactgcccaccatactgtctttttatgtgaatcaacaaagccacatttgtattggtgctttctttcctttcttttttttgaaatatgagatgcttcatgaatttgcatatcatctttgtgcaagggacatgctaatctctattgttttaattttagtatatgtgctgctgaagtgagcccat

SEQ ID NO: 12 - DNA, 701 bp, Homo sapiens
cggagtttcaccatgttagccaggatggtctcaatctcctgacctcaagtgatccgcccgccttggcctcccaaagtgttgggattacaggcgtgagccactgtgccggcctaaatgtctgttgtttaagccacccagtccatggtattctgttatgacagcctgagctggctgatatagtcagttactgctgtaggctgctggggatggaggtgggaaggtagagtaacttcccaggtgaggaggcttctgtgcagttgaggacagttatctggacataggggtaactgtgagccattaacagccatcatacacagcacccaggggatgggtgcattggccagcatggaggggagctgggcagggtccaacagcatctggcacactcaagtttcccttggtctcctgactgtcttcctggtttcattgctgggactccagccccctcctccttggtcttggcaagacagatcagagaggagcgagcactaaaggttgtgyggggtccttcccagcagtgccaggccagaagtggccttgtggtgtttgtggaatggccccacctcatccagacccaagaagcatggtcagtattaggattttccactgaccatcagcccaggacactgccgtttgccctgagtgtcatatgttctttggaaatctcagcatctctagaggtcaaaggttttctaacttgc

SEQ ID NO: 13 - DNA, 601 bp, Homo sapiens
gaaccaagatcacaccaatgtactccagcctgggtgacagagcaagactctgtctcaaaaaacatacaaacaaacaaaaaacccaaaaacctagtttggtttacaaactgccccaaaacaaacttctgaacataatcttttcttaaaggtatgtcttgcatataagaaaggtttgtatatttgtactttttttctgactttgttttattctcagcaatctgaggagtttgttccctggagcctatttataatgatgcttcaggttctgctgcatgtcccctttggctttcctcctgaagcrgagaagcatttgcacagagcacaccaggtgttactttcatattctcagacatactagactctattaacaaaaacactaaatcaaaacacagcaaaattagtgatcatcgccggagtatgcagcccaaactccttacgacagctcgggaagtgccctgtgtctctgttctcactgtctacatgattggacaatttgatactgatagtaattgacattttaactattagttggtgttcacataaattatagaccaaaattcatctcacttagtcagcatcatgtcaaccatttcctttggct

SEQ ID NO: 14 - DNA, 3563 bp, Homo sapiens
tacattgtgattttttaatcttttttcaaagaattaaaactgtatcacttgggagctatcaacctaatacactttctttctttttttattacactttaagttttagggcacatgtgcacaacgtgcaggtttgttacatatgtacacatgtgccatgttggtgtgctgcacccattaactcgtcatttaacattaggtatacctcctaatgctatccctaccccctccccccaccccataacaggccccggtgtgtgatgttccccttcctgtgtctaagtgttctcattgttcaattcccacctataagtgagaacatgcggtctttggttttttgtccttgcgatagtttgctgagaatgatggcttccagcttcatccatgtccctacaaaggacatggactcatcattttttatggctgcatagtattccatggtgtatatgtgccacattttcttaatccagtctatcattgttggacatttgggttggttccaagtctttgctattgtgaatagtgcccctataaacatatgtgtgcatgtgtctttatagcagcatgttttataatcctttggttatatacccagtaatgggatggctgggtcaaatggtatttctagttctagatccctgaggaatcgccacactgacttccacaagggttgaactagtttacagtcccctcaacagtgtaaaagtgttcctatttctccacatcctctccagcacctgttgtttcctgactttttaatgattgccattctaactggtgtgagatggtatctcattgtggttttgatttgcatttctctgatggccagtgatgatgagcattttttcatgtgtcttttggctgcataaatgtcttcttttgagaagtgtctgttcatatccttcgcccacttgttgatgtggttgtttatttttttcctgtaaatttgtttgagttcattgtagattctggatattagccctttgtcagatgagtagattgcaaaaattttcgcccattctgtaggttgcctgttcaccctgatggtagtttcttttgctgtgcagaagctctttagtttaattagatctcatttgtcaattttggcttttgttgccattgcttttggtgtttttagtcatgaagtacttgcccatgcctatgtcctgaatggtattgcctaggttttcttctagggtttttgtggttttaggtctaacatttaagtctttaatctatcttgaattaatttttgtataaggtgtaaggaagggatccagtttcagctttctacatatggctagccagttttcccagcaccatttattaaacagggaatcctttctccatttcttgtttttgtcaggtttgtcaaagatcagatcgttgtagataagcagcattatttctgagggctctgttctgttccattggtctatatctctattttggtaccagtactatgctgttttggttactgtagccttgtagtatagtttgaagtcaggtagcgtgatgcctccagctttgttcttttggcttaggattgacttggcaatgtgggcttttttggttccatatgaactttcaagtagttttttccaattctgtgaagaaagtcattggtagcttgatggggatggcattgaatctataaattactttgggaggatggccattttcacgatattgattcttcctacccatgagcatggaatgttcttccatttgtttgtatcctcttttatttcattgagcagtggtttgtagttctccttgaagacgtccttcatatcccatgtaagttggatttctgggtattttattctctttgaagcaattgtgaatgggacttcactcatgatttggctctctgtttgtctgttattggtgtataagaatgtttgtgatttttgcacattgattttgtatcctgagactttgctgaagttgcctatcagcttaaggagattttgggctgagacgatggggttttctagatatacaatcatgtcatctgcaaacagggacaatttgacttcctcttttcctagttgaatgtcctttatttccttctcctgcctgattgccctggccagaacttccaacactatgttgaataggagtggtgagagagggcatccctgtcttgtgccagttttgaaagggaatgcttccagtttttgcccattcagtatgatattggctgtgggtttatcatagatagctcttattattttgagatatgtcccatcaatacctaatatattgagagtttttagcatgaaggttgttgaattttgtcaaaggccttttctgcatctattgagataatcatgtggtttttgttgttggttctgtttatatgctggattacatttattgatttgtgtatgttgaaccagccttgcatcccagggatgaagcccacttgatcatggtggataaactttttgatgtgctgctggagttggtttgccagtattttattgaggatttttgcatcgatgttcatcagggatattggtctaaaatctctttttttgttgtgtctctgacaggctttggtatcaggatgatgctggcctcatgaaatgagttagggaggattccctctttttctattgtttggaatagtttcagaaggaatggtaccagctcctccttgtacctctggtagaatttggctgtgaatccatctggtcctggactttttttggttggtaagctattaattattgcctcaatttaggagcctgttactggtctattcagagattcaacttcttcctggtttagtcttgggaggatgcatgtgccgaggaatttatccatttcttctagattttctagtttatttgcgtagaggtgtttatagtattctctgatggtagtttgtatttctgtgggattggtggtggtatcccctttatcattttttattgcatccatttgattcttctctcttttcttctttattagtcttgctagtggtccatcaattttgttgatcttttcaaaaaaccagctccagaattcattgattttttgaagggttttttatgtctctattacactttcaacttggagggaagtagaaaactttgtttaaagctgaggactcaacagtctctcaggtagttgactggctgtggtgatttgtgaactcagaagcctatggataatgaatccaatctttmtttctaggtcagaaaactacatgtatctggtcactgaaataaacgtatggtagagtgaaaagaacatgtgttttagaaacaagacccattgacttgggtttcagtgctgactaaacatacattactctgcagaatcttcgtcacattacttactcaatctctctgagcctcagttttctcatcaataaaatgaagacaataataatacctgatatgtatattttatgaacaaattacataaagcacccacctgaaacaacttatagataacaggtcctcaacaaacctttgtttctctcctaattctctgagaaaggaaatctgggagcaataacaatgttttagaagcatcctaggtctcaaacc

SEQ ID NO: 15 - DNA, 501 bp, Homo sapiens
tagggtttttctccagtatgaattatctcatgttgagttagttgtgaaagcatgcaaaatgatttgccacattctttacatttgaaaggattttttccagtatgtcttatcttatgtctctttgcatttgaatatttatgaaagactttcacgtatttgtcacactgaactattttgctctgggtagttgtcacacaccggttaagtcccttgtgacctcctttgtgcaacttatgctcatccacactttyacagcctttctgatatccacattttccatatcttctcagtatcacttgttggaaagaattttttatatattgctctggcctaaggtctttggcaaaatgagaacacatagctgaaagaaataaaaataacaaattattccatttactcaactcagattaatatttacaaatctaacttataccaactatataaacaagatgacatagcaaaatactacagatcctaaatcctttatagacatataaatgt

SEQ ID NO: 16 - DNA, 701 bp, Homo sapiens
agataactctcaattcttcatgttcatccttgacagatctcccaggctctggtcagagctgtggtcaggtttccaagcttgggagtgtctccccttccctcaaatgcagcctgttggaaactgaagccttaatcaacatccctcctatagccttctttccttttcttttcttttttgtttagagacagagtctccctccgttgcccaggctggagtgcagtggtgcaatcttggctcactgcaagctccacctcccagattcaagtgattctcctacctcagcctcccaagtagctgagattacaggcatccgccaccacactcagctaatttttgtatttttagtagagacggggtttcaccatgttggccaggctggtctcaaactcctgacctcaagtgatccccaccccttggcctcccaaagtgctagggttacaggtgtgagccactgtgcccagtttaaccttcttttcaaactcaatattctcttctaacaaygttgctaaccatcttcccagttatttctttccccatatcctgtgagtagcctggtcctatagaatctgccactggattttctcagccccacactctctccagaaccccatgtctgcagtacctttcaccaacagttgcttccaaattgggtcatctggctttagctttcttttcactccaaattaatcccctgaagccaa

SEQ ID NO: 17 - DNA, 601 bp, Homo sapiens
agcaagtggtgactgaattgaaaagcaactctgagttcaaaagatcaggtgggaccgatggcagcaaacaatccattgaattgctgttagattgaactgtcttcagaaagaggttgctgtggcttggctggggcacagacactgcaaatccagagacagaacttcctgggctctggaccatgatagataattaaagatcaggagcagcttggctaaaagaaagccaatagtcaaggaataatttcaactggaaaagaaaaggatagaggcccttctttaaggaaggtagaaaaacatcccrggctttgcagaaaaaaataacagcaggaagaagctttctttacccttttttttttttcctgcaaatcatgattggttgagtcccccagcagtgtgtggattgataacgaaggggcagaatgacaagacagcctcaatggcaaaaccttcatgaaagccggcagagtgaaaaaacagctctgggtagattcctgcaagggctaggactgaaaccttcatgtccacaaaatggtctattgtaaaccttgagaacatcttgctaagtttatccaaagtcagtgtttcccttaactggttgcta

SEQ ID NO: 18 - DNA, 601 bp, Homo sapiens
ttctgactcagtaggtctaagtccgtgtttctaataagctcccaggtgatgttgatgctactggtcctgggccaagcacacactttgaggaacctaagaatctagtatattcgcaggaacgttcagtgggataaaattaaaggctttcttgatgtgtaaagagattaaatgtagtacagttttccctttactccctttatcaacattactttgccatatatcaggaagggaaattaaactgttctttctaacactatttgggcttgtacaaccatgcaaattactctatttttggcccttyaaggacatttcaaaatggtgatgttctactttcttttcggggtgttaacattaagctgactcatccaattttgcttagacaaaattgtgaaagtaaatggtctttggttttcctggtttccaggttaccttatgatgtcttgccttttctcctcaatgatggctctacacttgttctaattagagagcaaagaaataataataatagtgtaagacagaggggaatgcagtattaaattcctattatgtgacatatactgctgcaagtgctggagacacagtgataaaacagaaatgtgtc

SEQ ID NO: 19 - DNA, 607 bp, Homo sapiens
tttcttttcggggtgttaacattaagctgactcatccaattttgcttagacaaaattgtgaaagtaaatggtctttggttttcctggtttccaggttaccttatgatgtcttgccttttctcctcaatgatggctctacacttgttctaattagagagcaaagaaataataataatagtgtaagacagaggggaatgcagtattaarttcctattatgtgacatatactgctgcaagtgctggagacacagtgataaaacagaaatgtgtctaactgccttttcactttaatccagtgagatgcatattcttttcatgatacgaatgagaactctgtgaggtaaataactcagcagtgccacctggtaagtggcagagcagagctatgaatccagttctttttgaatccaaaattcttgctaaagctcctgaactcattctgaacaggaatttgcctacccccttaccgcaataaataacttagaggttttgcattttttaccattcaaaaaatgttctatttctctttcttatcctttcagaataaaactgctgatgctttagtgacaggtttcaacatgttttgtaaaaaacgatccaaggaatt

SEQ ID NO: 20 - DNA, 601 bp, Homo sapiens
cacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttrcactccgaagactccagattccgagagttgcggaaacgctacgaggacctgctaaccaggctgcgggccaaccagagctgggaagattcgaacaccgacctcgtcccggcccctgcagtccggatactcacgccagaaggtaagtgaaatcttagagatcccctcccaccccccaagcagcccccatatctaatcagggattcctcatcttgaaaagcccagacctacctttgagcctcagttgccccatctgtgccctgggtaggaatatcctggatccccttgggtctgatggggtag

SEQ ID NO: 21 - DNA, 726 bp, Homo sapiens
cagctactaacgaggctgaggcaggaggatcacatgaacccaggaggttgagggtgcacagtgagccatgattgcatcactgcactccagcctgggtgacaaagcaagaccctgtctccaaaaaaaaaaaaaaaaaaaaaggaggggactggacggatgtcattaaatttaatacaatctactacattccatcctcttcagttcccctttcctctgccttttcagcggcatggacaagcctttagaacatgtttctcaggctcctatgtcagctggcttctggctgggttcagctaagggaagccgccccagagacaggagagtgtgaggctgggaaatgccagagtatttttccccatccctcactatctcagggagcttctcctacagcagctctattttctttgtggttctagctcccatgtgataggcctgctatagtttctgcttctgctgagtaatcccagatgctacaaccttaagagtcatataatctgatcyagtttcctaatgtgtaaaacagggagagaatctgctcacattatctatttcaagaaaaagaaaaatccctggttgtctagtggttagggggaaaaaaacaaagaatgtacattaaaattgtaaacaataaagtgctggacaaataaggaatttaagggtatacgaataaggcaatatggtgtagcagaaaagaaaacaggactggggataataattcttggttca

SEQ ID NO: 22 - DNA, 601 bp, Homo sapiens
tcgctatggtctataaaatatggaaaccagaaactactgtgtaatgttaccccacagcattaaaccacttctcagagcagttaagctctaattatgaaaggaaacagatgttgccagttctcatctctcacaattcccacatctgtcaaggccctcctccatgggttagagtggggagatcctcaggggagaattggccattgggtttctcaatcacctcctggaattaggatcagtagtagaaacagagaggtgttttatgaaggtcaatttctagttacctcatgcaccagcccttaamtgacagcagggagcaacacttctgatttgtaccaaggcccctggagttactcagctgtattaaactattaggaggaagaatgaaaatcgaaatagatcgcatccatttgaggtgggctgctcaaaatgcgttcactgggagtgttaaattcaatatagatgacaaggtcggatgagatccactgcctcatcccaagcagcttaacgttcagctgctcaccacaggatattactgcacacttcattagattatacttctaacctgaaacaataactgttaaattccaaactacatcttagt

SEQ ID NO: 23 - DNA, 401 bp, Homo sapiens
aagatgttattgcttatgagtcttacttatagaactagatcctgttaaattttagtcttatgtatcgagggaaagaattattaaacttattttatttgactatgtcaagaatacagagctctcctaggattaattagtggttgacagtattactaatctttaccagttgtggcaagaagggtggttttatttgagtacacratgagtagagctttttacaaatgaaggaatgcatgggaattctaaaggcattatatttggactcgctaaagaattctgtgtttatggtaaaatgacttcaattaagctcatctaggttttgttctcatatgatatatttttgaaactttttgtttattcatttgccattgtgttctaagtaccacatcgagtacaacagg

SEQ ID NO: 24 - DNA, 701 bp, Homo sapiens
tgccagtctccttcatccagctgaatggtctagttcttctacttaaatttaaattataagcctactggaaagtatttttgtccaactgagcatattcatttcgttattcaggtacttttgccccttattaccaggaaattaaatgtaaaaatagtgtgaaacaaaatgattggtatacttaaacacccccagacacccatycacccacacattttctatagagggttaatgagaagtgaaaatgtttttctgtgctatccattgtaatagttgctagtcacatgtggttattaatcacttgaaacataactagtttggttgaggaactatcaattttaagtaatttaaattttaatagccacatgtgactagtggctactgtacaaggcagcagagtgtagataattttcaagggtccccgacccccagggcacgaaacagtactggtccatggcctgttaggaactgggccacacagcaggaggtgagtggcaggcgagctcccatttaccacgtgagctccacctcctgtcagatcagcagtggcattagattctcacaggaatgggaaccctattgtaaactgggcatgtgagggatctaggttacatgctccttatgaaaatttaactaatgcccgatgatctgaggtggaacagtttcatcttgaaaccatcccccacaacctgtgttaaaattat

SEQ ID NO: 25 - DNA, 665 bp, Homo sapiens
actacattgtacccacctcaaaaaaattatttgttttttcgacaccatttcactctgtttcagcctgttgcccaggctggagtgcaatggtgcaatctcggctcactgcaacctctgcctccagattcaagcgattcttgtgcctcagcctcccaagtagctgagattacaggtgtgagccaccatgcccagctaatttttgtatttttgataaagacagagtttcatcatgttggccaggctggtcttgaactcctgacctcaggtgatccacctgcctcagccttccaaaatgctgggattacaggtgtgggccacccacctggcctcaaattttgaatagctagaatattggcagaatagcaaaggcttaaaacctctttactatcaaatttaaacattatttagtcttaaggaaaccgcaatgaatgaaaagacttttttctgtaacatttattttctgayatggtaaaatatctaggattcaggtgtgtcttctgaaggaacaccagaacttgaaaggcacccctgtctttctcttggcctggctccctgggcacctcacttgaaggagagagctgtttaggaccggcctcgtgtaggtgctacagggggacccaccatgcagaggtggatagaatgcagtttacagaggatggagaaca

SEQ ID NO: 26 - DNA, 401 bp, Homo sapiens
gaacacctttcagatactctggtttccttccacagacaccacctcagataattttggatacaacctactgacgttcatcatcttatacaacaatcttattcccatcagtctgttggtgactcttgaggttgtgaagtatactcaagcccttttcataaactgggtgagtattaaagcagagttgaatcactattttccaaygctatttcagagcctttggcatttaattgagcactcaaaaaagaagaactatattcatttaccattaggtccaggggcttaaactggccatacaaagaactggccagggatcaccagtgagggtgtttttggggaatggaagggcagtggtacctattcaaggcgttgttgtgaggattggtcagagaatggggtggaat

SEQ ID NO: 27 - DNA, 952 bp, Homo sapiens
aaaatctgtgccatttccactgtttaaaataggtaaaagcatttggtttgagaattttggtcagtaatttatttcccctctctcctcttcccaaattctgttaactcttattgtctgctgtgaacttggaagcatttatgatttttgattaaaaagaattaagcatcaggatttgtgagacaaattaaaactcaaccagaattggaagagacccaagacgtgacaagtaagtactgtgtgkgtccctgaattggattctggaacagaaaagacattggtggaaaaactggtgaatcttgaataaagtctgtaattggattagcattgtgccagtgttactttcgtagttttgacaattgtgctatggtgatgtaagatgctaacattgtggggaaactggacgaaggatagatgtcaactctattatttttgcaactcttctataagtatacagttttcggccaggtgtggtggctcacacctgtaatcccagcactttgggaggccgaggcaggcagatcacctgaggtcaggagttcaaggccagcctggtgaacatggtgaaaccccgtctctactaaaaacacaaaaattagccaggcacggtggcgggtgcctataatcccatctactcaggaggctgaggctggagaatcgcttgaacccgggaggtggaggttgcagtcagcagagatcgcaccattgcactccagcctgggtgacgagagtgaaactctgtctcaaaaaagaaaaaagtatactattttctcaaacaaatcttaaaatcaactagaagtatctgagagaataggaatgaaatgctaaactcttgtttttctgttgaatactttagtaaatgtttggatctttaattggaaacatacatgattttgaagggaggatggtcagaagaaaaaaggatcagtatcttcatatgtttgttgtgatgagt

SEQ ID NO: 28 - DNA, 734 bp, Homo sapiens
ttctctgtatcacattttataaatattgttcctttctctcaaaactatttttttaagttttcacaaattacatgaggcttttattttattttattaatttataaatttatagtcttatttgtgagtcttgggattgatttattttattctaagatctgtaagtgaatgcaaaggtaactttttttttaatttgagtatttgagttatgtttctaattagagattggactgttartttgatatgcagtgttctctgtatcacattttataaatattgttcctttctctcaaaactatttttttaagttttcacaaattacatgaggtttttattttattttaatttattaatttataaatttctagttttatttgtgagtgaataaagttctaatgttcgatagcagactacagtgactatgcttaatattttttatactacaaaataactaaaatgagaacttgaaattatactaatacacaaaaatgataaatattcaagctgttgtataccccaagtgctctaacttgatcattacacattctatgcatgtaataaacacatctactccataaatatgtaaactattatgtatcaacaaacgaagaaaagagagacatgaaagaggtgatttctctcttgaccatgtgaggatataataaaaagatggctgtatacaaaccaggaaaggtataattatagttactctagtctgctatcaaacattgtaactt

SEQ ID NO: 29 - DNA, 601 bp, Homo sapiens
caggaaacggagattgtttcatttgacacaagctccgttttgcaggactgagtacgggagggcagagtgtggggagggaagtctctgcaaaccagtgactgttttggcaaataggaggagtgaagaaacaaaaggtggaaactctgagagataattgtacacgggtcagacaaatccctgtaaaatcagctttttgtaccttcttggtagcatccgcgaaccaccatcagcctctagtgtgcgcatgcgatggaggaggggacactacaagaacagggccagggctcttttcagatcaccrtcatctcagggatgttctgtgctggtatttcaaaatcaccatgcgtcatatcagactgatctctagttagataagtaacagaaccaagtgggctgatttggaacaacaacaaaaaactaatgtatttcaccgatcaagataacaccaatgctgtgttgttttcctattgtatcattaccatgattattattgttatttaaaagtctgctacctgctcttgcttttaccatccagtcgcctcctagaagcggagaaataattacgtgtaaatcccatcatatcactgctctcacagctccc

SEQ ID NO: 30 - DNA, 1294 bp, Homo sapiens
agaattatgagtgacaacaacatgggttatgccacaaagaatcagctgtcaaaaagaagaggaaacaaatatatcaaagtaggccagaaaatttgctcatcagtgctgtctgctagaactttctgtgataacagaaatgtggtatgtctgtgctgtccaatatgatagccattagccacatgtggctacagagcacttgaaatgtgactaggagatgaaggaacagaatttttaatttaatttaagtgatatttaaattttttaatttaaatagtcatatgtaattaatggcttccatgttggacagcacaaatctccttcgtcacttaagggttattgcctattcattagtgggaagaataatatatttatatcaatgatatgttaattatatattaaaaataaataattatagataattatagttggcagaactggcctagttattttccccacaacaatgctgatcttcctatgatgagtccaagattttgcaacatttggcttttgctatggtttggatatctgacccctccaagcctcatgttgaaatctgatccccatgatggtggtggggcctaatgggaggtgtttgggtcatgggtacagatccctcacgaatggcttggtgccatcctagtggtaatgagtgaattctctttttagcaaccataagagctggttgttaaaaagagccttgcactttcccacctcacttgtgcttcctctcttgcaatgtgatctctgcacatcagctctccttcaccttctcccatgagaggaagcagcctgagggcctcaccagaagcaggtgctggtgccatgcttcttgtacagtctgcagaactgtgagccaaataaacctcttttgtttataaattacccagagtccgctattccttcctagcaacactaaatgaactaagatagcttttaattacaatggtggcatctgttactactctgcattctcatcttcattccaggtaagtaaattttaagttttggcttctcttagttccaagacacaacataagtcattccctgttttkgggaggagagagtcaggatggagaaaagaataaaatttattgaaaatttaaaattaagataattaaatatgtgaactaaaagtgagtaatataataccttctcccagaaaagatcaggacgggagtgttacaaagctttgacatttcttaaagtgcactctgctgcattttgtgtgtggtctatgtgtaatagctcatcttcaacacacacacacaaacacacactcaagtgtgagaataaa

SEQ ID NO: 31 - DNA, 861 bp, Homo sapiens
atgtcacatgaattgacctatttaatttccctgatcatttactagaattgccataatattgatttgaaaaaggagacctgagcacataagtgatcaaaaacatatgagatgaatgagtaaatgaacggagcttgcatttgagaggctgaacacattggcagtgacatgaagcacatgagaatgacagcactaaacgcagcrcgcaacctgggaaagaggctgaaaaaatacctactcagccacggtaaagggtttagactgtaaccaagtacccacctttttctaagagaaagaattacttttttaaaaaatacttttttcttcttttctgtttcctccttttcccttgttccccacttcctacttagctctttagaaatgcaattataacatttacctttccttcaccagacactccctgtagggcaagcttatctgtgtgcttacttggaagctcttgcttacttagaagctccagagccagacgtctcttcgaccaggagactgcctcgagagataacaaattataacctaaagtatgcccatgatgaaactcactcccacttggagagtatctcaagactctggccaccttaccacctagttctgcccgcgagggcaccagctcaaccacctggtagataaagcaccaaagcaagtcattgagacccccactggctcaccccctcccctgcatgccattcatgtcaagtccccctttgaaaacccttgcttttttgccccaaaagtgaagcagtacccttaaaggcagaagcctgtacttctccccctcaacaaagctttggaataaaagttacaagatcaagttttctaaaactttttgaaactcaatcattcaaa

SEQ ID NO: 32 - DNA, 501 bp, Homo sapiens
agctcctggggttagggcttcgcctgtgaatttgaggggacccagtgcccttcctcgaaatgtcgtgttgactggcagtggctctttgttccgggtctctgagcatgactgttagtgataacctcgcataccgccaaaaacaccagcccctgaggggtggtgcagaaacacctgtggagggtgcccaggccattgggcatcgccttaagcaggtgtgcagggcaggaggggacgagagttctgtaactggmatgcacgcaccattctgagaagccgcatgagcttaaagagaggcctcaaacctgagaggcgtccctggaaaccagggctgctctggagtgcacaatttttcccatttttgtggggttgagccttttcaataagatttcaagagaataaaatccacaggccccagggaatttgcatacggctacttaacatcaattctgtatgttttttaaaaaataaagaaataaacacatccacaaacttccccatcca

SEQ ID NO: 33 - DNA, 1061 bp, Homo sapiens
cttcttgattaaaaagggcctggaatagttagaaagaatgaataagacctagtacttgatagcacaacaaggtgactagagtcaataataatttaattgtacatgttaaaataactgaaagagtataattggattgtttgtaacacaaaggataaatgcttgaggggatggatactccatattccatgatgtgattattatacattgcatgcctgtatcaacatttctcatatacctcataaatatatacacctactatgtacccacaaaaattaaaaataaaaaaattttaaaaagggccggagtgagggtggttggtgattggctattcctttatgccaaagacttcatattcttcttcatatgtaattctcacaataatcctacaaactagttattaatttctgtattttatgaggaaactgaagcttaggggaataaacttgacccagttacaaggtaatactactcatcttacagcatttatggagcccttacttgatgtgtcaggctttgtgttatgaaccttaaacctattaacttgtttgagcctcaccmagctgtggaaggtgggtaccattattacctccatcctgcagatcaggaaatggaaacttaagagaggcgcagcaacttgtctcagcttatattccaggggctggtacatggcagggcccatgtttgaatccattctctttatttattttttgaggcagggtcttggtctgtcacccagggtctttcttgctctattgcccatggctcactgcagccttgacctaccagactcaagcatcctcccatctcagcctcccaagtagctggaccacaggctcatgccaccatgcccagctaactaaaaaaaaaaattttcatagagacagggtctcactatgttgaccaggctggttttgaactcctggcctcaagcgatcctaccaccttggcctcctaaagtgctgggattacaggcgtcagccacctctcccagtcctcttctgctttagagctcttgccctactattcaaatccacgctttggcatttctttgcctactttgg

SEQ ID NO: 34 - DNA, 501 bp, Homo sapiens
catttctgtcatgtcaataaaggtctgaattttcctatgaaaagaaacagactcctattgagccacaaagccaattctaactacacactgtcgtcaagagaaacacacttagaacaaaacacctatacccagagagaaccatactcagtaatgaggtgccaaatgaattcagtttatttcccataccttcccaatctgaatgaagtgagggaaggggcaaagctagactataattagctgggtagaggagrcttaatgagctgtactctgtaagtaaatctactgatgcttgtgttctatttgttttctgctaaagatctttttggaaatttagacctcccttttgctgacaatagggccagcctgtctgtgtgccaagccctgtctgccctgcattctccctccccaggcctctgagctgcctctagattaaagagacacaggatgcttatggctgactgagaaaaataagactagtggtgatctgatacaacaacagtc

SEQ ID NO: 35 - DNA, 603 bp, Homo sapiens
atagtgcggggagagaaagctatgggagactagagaagggaaaaatggttgggaacactggtgggggggtgaggatcatttcccaatcaccttatatgaccatgactcagaacagtgcttggcatcatgtgtaatatatattgttagaaccgaacatctagttcaggcaacaattcataaaacgttgcagggaataaactgtgggaaagggtctttgacaaagaataaatttcagttgaccaaagactgaattgggagktgtacttctaaggcaggggcaataagatgaggcccaataacaaaatccaaaaagaacatggtctgagcaattgtacgccaggcatttcaggggccacataaatcatgtataggagatttgtgaaaacctaggctgggaaaccagatggtggtcatattgagaacactcctacctctgtgcaaggaaagaagagcaattgctgctgtttatgcaagaaagttagagcatcagagtttgtaaagatgaaactgatgacactgcaaagcaggagccaataagactacaaaaccaaagcaacctgataattacaattattaatataaatttaaaggagctgggcagaa

SEQ ID NO: 36 - DNA, 716 bp, Homo sapiens
tgatatggtttgggtctgtctgtgtcccttcccaaatctcatgttgaattgtaatccccaatgctggatgtggcgcctggtgggaggtgtttggatcatgggggcggatccctcatggcttgatgtggtcttcgtgttagtgagttcttgcaagatatggttatttaaaactgtgtagcacctgaccctctctctcttgctcctgcttttgccaagtgatgtgcccgctcccactttgccttctaacacgagtaaaagctccctgaggcctccccagaaccagatgccactatgcttcctgtacagcctgcagaaccgtgatccagttaaacctcttttgtttacaaattacccagtctcaggtatttcctttttttttttttttttttttggtcttaaactccaagcctcaagcaattctcccaccttggcatcctaaaatgctgggattacaggcatgagccaccatacccagccaggtatttctttatagcaacacargagtggcctaatacagagggatattgccaatgatttaatagtagtcttcttttctgtatgtcactctgcttgactaaccttaaacagtctctttaaataatttgtaagattcacctaactacacttttcatattgtccttcaatccttgcacttatggcataaacatgttttatgattattatataggtatttaaataatgcatatgggtcctat

SEQ ID NO: 37 - DNA, 601 bp, Homo sapiens
ccttccagctagagatgtattagaatgtctgccaggatccccaattcctggtccagtgctgttgctattaatggcactactcatcaaactaagtcacctcctctatgacctacatccttttttgccaactattttgtgttaatggaatcactctatctctgcaatgaaatgcaaatcattattcttcaaataacagccgtagtgatcctgaagctctttgtgtaggcttccttcatttatctttccctataatttcagtaatgagcataaaaggatctagacaaaaatagtatgttgagaratgccaacataaaacccagcagtgactcatttcacaagctatcttttattttccctattctcattccctgggtcatcgatgaacaaagttgccagccttgcttcatatgttacctcttgcatggattcagcaataataattggaactgaaaataaatgtatgtccaggaatgaatgatcagccctttattcgccagatgattcaagatttccagtttctccattgtcatttttatactgcatatgctctttgttctctgcaatgctttattgacctctttcaggttgtaaagaagcagat

SEQ ID NO: 38 - DNA, 601 bp, Homo sapiens; identical to SEQ ID NO: 10

SEQ ID NO: 39 - DNA, 801 bp, Homo sapiens
caagaggcactgttaagggacggcctttctgagtaaggtgtcacagctggtagcgtaaaggcgctgggtgcctgtgggtggataggggagcatgtataagagagggtaaaggagacgggcctccttctagtcctaaccactatgctcagtaactgatagaattcatcagctaggacgatgaacaagtagtgtgatgtacacctgcaaattaactttgaattcagtacttgcttaactttctctgtgtcttataatcccatgccttgtcattttcagataatgctaggtctctgagccattacacttgaagagctatggggaaatctagagtgttttatcttgctgaatgccctttcttcttttctttagattacatgaagcagatgacatttgaagcccargcctttttagaagctgtgcaattcttccgacaggagaagggtcactatggttcctgggaaatgatcactggggatgaaatccaggtaatatggggttcaaaattttggattctctctgtgtttgggtatttgggtgatacgaagtcagggtcattttattaaacaatcaaacagaaaagttgcaagtatggaagatgtggttccaaagaccatctctctcatgtctgcaagcattggtcaggaaactactgggcacctactacaaagaccaccagactgggcatgaaatagggcagccaaaaacatgacaactacagtggaaagagctgcctccactgttttctatgaaaagaattactctttcaaatgacctcgtgcttccagactagaatcaagtgtg

SEQ ID NO: 40 - DNA, 601 bp, Homo sapiens
gggaaatttctctccctgctaattgctgttagcacccatcaggatcacggagaaagcagtgtcccagccagaggggccttagggctgagctctgtcaccaggagacggtctctttaggaggatgagggacccataaagggctgcacatgcagggcatgcccaccatggaaatccaaggagtctagaccagcagcttccaaactgtgtcccatggagccacagggccctgggagaggcctgagaaactggtgtggaggtccctgggtaggggggttgatctgtggggtgctggattctaccracctcttaatattactagccccattgatctattttatatctaaatcagcgctaaagggtttatccaagaagttctgctaattcaaaaagaaagatttgaaaactgcagatttagtccagtcctcacctttgacaggtgagtagacacagcctggggagtgggaggtagagagaggggaggtactacaaggtcacatagagaatccagttcagacctgggccatgcgcacagacctcccgattgcacaggcacgtttctgcctttgtgtttttcactttccacatcccacatctgcctgtt

SEQ ID NO: 41 - DNA, 703 bp, Homo sapiens
ctaatcctacatataaaaaaggagggcttctctgattgaagatgggcaaaggactctctggggctccctctcactcaccgggacagagagccccaggtaccccagggcttggaggaacggtgaaaaccaatcacctgaattagtggtcattttcatagttcagatttcaccagctattaaaaccacaaaggaaatggacayaccaaggcaaactttttattttgcccagaaaggacgcaaaaacaataacaatataagcaacaatcaccactgccctccaccacaaacatgattttgtaaaagaaagggcatttttaatgcttaaaaaaaagtaaggaagactacctctgattataaaatggtactttaaattagatttagctttatttttatttttttctttgacacggagtctcacactgttgcccaggctggagtgcagtggcacaatctcggctcgctgcaacctccgcctgccacgttcaagcgattctcctgcctcagcctcctgagtagctgggattacaggcgccctccaccacgcccagctaattttttatatttttagtagagattgggtttcactatgttggccaggctggtctcaaactcgtgacctcgagatccacttgcttcggcttcccaaagtgctgggattacaggcgtgagccactgcacccggccagtttagctttattaaaaa

SEQ ID NO: 42 - DNA, 654 bp, Homo sapiens
gtccttatggatacaaccatcaatagaagaaatatttacaaatatatggggataacccactgcaactccagaatagtgattatctttggagaggaataaaaaggatgaaagggttagaatctagatgtcactataaaattttattttgacagaaaaagataagttacaagcatgacaaaatgttaaaatttgttaaatctggaaatcagataaatggatggatattattttatgttttgccagattgaaagattctattaaaaattaaatcaaatactctacatatgttttactatagtcatctgagtatattagcctattatgactagaactgaactgtrtcttgttttcacacattaactacatagttagaacattataaacacatatgttctctcattttcccacccaaaccaaggtaggataaagttctcagctttcctttaccatgacacgcccccaaccaaaacactactctcaaacattgctactttagcttaggctatttgtgaaattctcagccccatctaaaaaagtaaggagcaatctcctattgaatatcagattttcccagtgtcttaatgacagcacaagtgtcatgagttatatcctcacggtctaaaataaattttaaaagatgtttaacattcagct

SEQ ID NO: 43 - DNA, 601 bp, Homo sapiens
cttgtctttaaaatattatgggactcattttgttgagcaaataagtaatgtgctcttattcatatataaagatgatgtcactgtttcaacacccattgaacagtgtggctttgggctgtttttagtgaccgaaatacattataatggagaaacagtaagcattactaaggattaaatttgtgttgtttaatgttatcatatttactttaattgtattaatattgaagtccaattttagtgatcaaataatttgtagtcatgtttagtacatggcaattcatatatcctcttcataagagcrttttgactcaaattctgggttcagaattaactggctatgtgctcttagcaaaaaaacaaaacaaaacaaacaaaaataaacctccatgctccacagtttcctcatatttaaaaattggaattcactgagtattttatactacatcctaggtatattcataaccctatttatatcaaattgttcatcaatcaagagagaaataataccaacttcactgggttattggttagattaaataaaatggtgcgtacatggtgcacagctaactgtctagggtgtgatagatgcttcaaaactttt

SEQ ID NO: 44 - DNA, 635 bp, Homo sapiens
cagaatcttatcagccctcttcactccatctccttctttatgagcttctgttttctttttctcctcttgttcaatcacaaaagcacaggaatggatctatcaagtacaaaattcttatgtggtgtctgagagaggaaggcattcaggtaatgcctgtgccagtgttttcatgccatcaagcatatgcatgagaaagaggtgctgcaatgtattcaaggcttattgcatcctaagttcagtgggacctatgtaaagggatgaggtcatttaattctcataagaacattataacttgggaatatctccatatcttaaatgaggaaaccgagggccacagaagttattgacttgtctgagggtgcagctgggactagtactcaagtctgtctgacyccctggtttgtattcttagcttctatgagaggcttcctttctaatcaaaagttgcataaagtttcacattcccaaaatattcttcaaagcatcataagtaacttcataaagggcaaggtgtattcacttgaaggtgagtgtgattctgtaaaagactgaaattttactgatgttgattatggaataactaggtgagctcactggtattataggtgtaggtaaagaatgagatgtggattcaa

SEQ ID NO: 45 - DNA, 4950 bp, Homo sapiens
tgttcataaatgcttaagtaaaatgcttaaagcatggggtttgcatcataaaacaagatggttgttaacgtacagttgctttgtattcaatacttataccttttttttttttgagacagagttttgctcttgttgcccaggctggagtgcaatggcgcgatcttggctcactgcaacctctgcctcctgggttcaagcgattctcctgcctcagcctcccaagtagctgggattacaggcatgcgccaccacatccagctaattttttgtagttttagtggagacagggtttctctatgttggtcaggctggtcttgaactcccaacctcaggtgatccgcccgccttggccccccaaagtgctgggattacaggcgtgagccactgtgcctggctgtattcaatactttcttttccattgccatcacttttctcactctccttgcttcattttgaaaaacaaaaataaaatcctaagttcctgaaccaactgaacaagccccccttggcgagggagacctcagagagagtttacacatggagttcccagccatgatgaaacgggggggttagacaagctccccatgtcctctcccttgctaactgcaattagactttctttcctaagggttaaacagaaaccagcgcttttgaaagacttgccagactcccctccccgtctgcagtttcaacacagcaactgcccagcattcttccctgataagagatcgctgaggtcccgtgtggtgcctcctgcctgtaaatcccagcatgttgggaagctgagacaagaggatcacttgagcccaggagttcgagagcagcctgggcaacatagcaagaccccatctctacaaaaaatccaaaatattcaccaggtgtggtagtgcacacctgtggtctcagctacttgggaagctgaggtgggaggatcacttgagcctaggagttgaggctgctgtgagccgtgattgggccactgtactccaacctgggcaacagagtgagaccccccgtctccaaaaaaaaaaaaaaaaaacaaaaaagaaagagaggccaggcatggtggctcatgcctgtaatcacagcactttgggaggctgaggcaggcagatcacaaggtcaggagatcgagaccatcctggctaacacggtgaaaccccatctctactaaaaataccaaaaattagccgggcgtggtggtgggcgcctgtagtcccagctactcgggaggctgaggcaggagaatggcatgaacctgggaggcggagcttgcagtgagccgagattgcacccactgcactccagcctaggcgacagattgggactccatctcaaaacaaaacaaaagaagagaccacagatcatggagtggttctgaccagtctacagatgctgtacactgagcgccttcgtgtcctctgcttcaccttttgatgtgtagggcttcattgtgacacatttaaatgttaagtctctgcccaaagtgaacacaggatgcatataacatgctgtttgctgatgaggcatatgtatgttctctcttcatgaatattcatagctcctcccataacctgttcaatatgtatagtcacctgttgaatatgtatagttgaatatttatagttccgtataaattcctgtctccttctttcctccctccacgtacctgcttctggcttctccctgaggctacgcttcccagcctgtgggatggcatcctgtaggctgcaaccctttgtaagaaataaagctctcctttccaaatttgtgaacctcataattcttcagttgacattttgtgtaatttttttcatcattttctcaattttgggagggggactgattgtatacaaggctaggcaaaaagaagaaccacacggaaagcattatgaaaaatgctctgttgctgagaatggctcccttggttctgtcctckctctacaccaacatcttccaggttacacatccttccaaagggcaacaaggctctaatgtgctaccaaatgtacttggaccatcactggcacgcttttcttaaaatgtttttagaccgggcaaggtggctcatgcctgtaatcccagtgctttggaaggctgaggtgggaggattgcttgagctctggagttcaagatcagcctgggcaacataacaagatcctgtctctataagcatttttaaaaattagccagctgtggtggtgcttacctgtagtcccagctactcaggaggctgaggcaggaggatcacatgagcctaggagttccggggtgcaatgagccatagcttaccacaccactgcactccagcctgggtgacagagtgagaccctacctctaaaaattaaaaaatttaatttaattaaaatttaattcaaatttaatttaattcaaatttaattcaaatttaatttaaatttaattaaattaaaatttaatttaaatttaattaaattaaaatttaattaaaaattaaaaaaataaaaaaattaataaaatatttttaatttcagggatgccgatattaaatataaatgttataaatagcactcaaaaatgcttcattgaggccaagcacggtgactcatgcctgtaatcccagcactttgggaggccaaggcgggcggatcacttgaggccaggagtttgagaccagcctggctaacgtgatgaaaccccctctctactaaaaatacaaaaaagttagccaggcatggtagcaggcacctgtaatcccagctactcaggaggctgaggcaggagaatcacttgaacccgggaggcagaggttgcagtgaaccaagatctcgccattgccctccagcctgggcaacagagcaagactctgtctcaaaacaaaacaaaacaaaaaacaagagctggatttttctccaacatctcatcgcagagcctttgcatttgctgttgcctctgcccgaacacccttccccgcatgctgacatctgcgtgcctggcttcttcattttatttgggtatttactcaaatgtcacctgctcggtgaagccttctctgaccaccctctacttgtagcctttttcccacactctatatttacctctccccaccatttcattttttattgcacttctttttttttttttttttttttttttgagacagtctccctctgtcacccaggctggagttcagtggcacaatcacggctcactgcaacctccgcctcccaagttcaagcaattctcctgcctcagcctcccgagtagctgggattacaggcatgtaccaccacgcccggctaatttttgtatttttagtagagacaaggtttcaccatgttagccagactggtctcgaactcctgacctcaaatgatccaccctccttggcctctcaaagtgctgggattacagggatgagccaccatgcccagcctctctgttgcacttcttaacccctgactttgatcattcttggttttggtctgtctcccttcattagaacataagttctggaggcataggggatttttcctatgttgattatcttgaaactcaagagcctggaacagtgcctggcacatagtaggcacacaataaataattttcaactgccactggccttttttctgagatggggggtcttgttctattgcccaggctggtatgcagtggcacaatcatggctcactgcagcctccaacttctgggctcaagtgatcctcccacctcagcctcctgagcagctgggacaacagacgtgagccaccacacctggctatatatttttttcaactcaattttaaatggctacagtgattggtttagaaacagacatgtaatctaccagtcatgacggctcatgcctgtaatctcaggactttgggaggccaaggcaggaggattgcttgagcccaggagtttgaaaccagcctggacaacatagtgagaccctgtctctacaacaaaatttttttaaaaaattagctgagtgtggtggtgcatgcctgtagacccagctgctcaggaggctgaggcaggaggatcgcttgagcctaggagttcgaggctgcactgagctatgattgcaccattgcattccagcctgggtgacagagcaagacccatctctaaaaaaaaaaacaaaacaaaaaacgaaaacaatgtattttgttgttgttgttgttgttgttgttgttgtttttaagatgaaatctcgctctgtcacccaggctggagtgcagtggtgtaatctcggctcactaaaacctctgcctcgtgggttcaagcgattctcctgcctcagcctcctgagtagctgggactacaggtgcacaccaccatgcccagctgatatttgtatttttagtagagatggggtttcatcctgttggctaggatggtcttgatctcttgacctcgtgatccacccccctcggcctcccaaagtgctgggattacaggcgtgagccactgcgcccagcccaaaaacgatgtttttaaagaaaagaaacagatatgtaatccactttgagctagagagacaaaatgaggctttgacatgggagaaaggggtcctttctcttgcccttcacttgccctggaagcttgcaggctagaactgtcaccactgtcacatatcataaagggaaagcctgcttgagagtagccagcagaggaaagccagagagataaagcgaggtcaggggttggtgacatcatttggcctctgtatgcagccatacctgaaataaacatccagtggtgtgctggtaaatgtttaacaatcagctctggggtgaggggtaagagagccctgattcatagcatctgccaatttccatagtgtaaatattctcaccaggctgattgaactcaggcttgggagagattgcacacagtcagtaagctgctccatcatatcactgccctgacttcgagttcagtgaataacccaagatatgcccttct

SEQ ID NO: 46 - DNA, 601 bp, Homo sapiens; identical to SEQ ID NO: 11

SEQ ID NO: 47 - DNA, 601 bp, Homo sapiens
tggccactaagtcccttgacaaccctgcacatccttgtcttgttagatgccactctgagtcactaaaccagtgactcatgctcagcaagcaaggccatcttgagagctaaactgaagggaattttcaagaacaacgagtgaaaccctgaattgaaagggaataaggctttttattgtctgagcaaaaaaacaaaaatgaaaggaaaaaaacactgctgtcatactgtggattaaactttcccagctgcaaattttactctgaggaaaaactgggcaccaaaaaaaaaagcttttacactcycttggaacttttgaggcattttaattcttgattagttgataggtaagcagagacccagaatatatattccaaattaggcagatgaaatgatctttcaataacttgtctagagcttctgctagcttagtcattcattcccaaagagtggcttcacctgcaacaccacatttggtctttccgcatccagccaagcctctgtctggcttgctcagagcacccaaatggccagaaaagaggtgagaaggaaaaagaaaatgtgcttactgcggggaggggatagatgtttaggattagggctag

SEQ ID NO: 48 - DNA, 501 bp, Homo sapiens
tgtcttatttttaattgtttttttaaaccataactattttccagcttctcatccattccatcatgtgccattgaaatagtgtttccctcgaccatacactttcccccattcttttgcaaaaggggaggaaggattcattatttctctaatgtttacctacgaactcaggatgaatgattcaagatcgggaatgaatcttgaatcagggaatggatcattaatcagaagcctggttgaagaggaaagactartggacagggaggtaaggggcaggagtctgggttcgaggccagtaagtaatctctctaggtccaacgtgataagagcagtggttactaatatgttttggagcacagctcacttgaaaatgtaatttaagacatacaggataactcatctgatcacagaagattcagggacaccagaagtcaatttaagattctctagtgctgtctaaagactccacattaggaatgctgggctcccttaggtatgttccaa

SEQ ID NO: 49 - DNA, 501 bp, Homo sapiens
ggcagatttactatgaatgtcaagaactgcatatagtatttatcatcgtccactcacacatatttctaagagatactcaatttgttgaaagcccttttcttctgcttagtctcaaagaattacaacccaaggtgcactgaaaaagaaatcaatacagtataaaattcctattctctcaatatgcgcaaacgtgagaaaatcaacgttgccttgaagagagtcttgttacttcaacaggagctgtatctacraagaagagaaagagatgagcaaagatattggctctaaaatgtggtagcattcttccaattcatccacaaagaagagagccatagaattgaaattttcatcagtttgaggaacctagaaaaagtgccttctgaatatacattcacttttttctgtacataacaaaaacattgatttattggatctggttatagtgattaaaaatccaaaaatttttgcttccatttgatttatttaagtgatacctattcc

SEQ ID NO: 50 - DNA, 801 bp, Homo sapiens
  misc_feature (401)..(401): n is a, c, g, or t
agctgcataatcttctcaccaactctacgactgttgcattctagagattgaaaacatcaaaccaaacctgaagcactgagaggttgggtaaccagctctggtcactgagttggtgagggatggattcagaagaggtctccaattgtgacaccgcagaggacctgctcttggccactacactctaccagctctgcaagaaggtgagggagggagggtggagtgtgatgggctcttactgtctccattcggttttgtcttagataaagggaactgagtccttaggcccctccagcaggaaaggctcacaacagcagcccctccggccctggggtcagtctgtattcacacctaagggcaagctaagttgtgaatttgacagaggacttggacagtgtctctanttgtcccggttatagctgtggttagtgtccaccgatggatgaggagatgcttccggcagattgctctcttagccacaattccctcatttatggcagcaaagcaggccaacgattgcaaaagacagaaataaaacagaaaatcttggaatgagcatcatcctgtaaaaagcatagagtgacatcaataacgggtagacaccttctccccaccttcgcacaactgtaactttttggttgttaaaggaacagataatcatatttctaaagaaagaatgccatgattcaagccctggagtgaataaagactcatttatgggaggttataccaagaggaaaatagtaaacgggcacgtgttttagcccttttagacttgtaaatggtccacatgtaatgtgctcc

SEQ ID NO: 51 - DNA, 628 bp, Homo sapiens; identical to SEQ ID NO: 7

SEQ ID NO: 52 - DNA, 619 bp, Homo sapiens
atacatacataaaaatacagaatacctaaagtagtggacttaacttagtaggaaatatcttcctgcagtgctataaagcgttacattttagaagatgcattttttatggttatcattctaaccagttagaagagcaggatagcacttaattgaaaaatggatggccctcatctcaaatggacaaagtttattatgaacataactaagatacacaaggaatgaaattcaaataggagattcaagcagaaacaggtgtgcagggaattgtgttgaatgattytcctgtcgatagcacagtctctttaagtgaagtcccctatccctgatattctgaatttctctaaaccagacttgagcaaagctgttcagagaagtaccaaaaagtaaaataaaataaaataaatataacccacttataaggggatggatacactagagtttattcacaccttggaatactatatggtagttaaaaatatgtcagcatggataaatttcaaaaacataatgttggaagcagtggtgattgaaggctcccatcaaaaagatccaaaatagcgtgcaaatcctgcactggcaaccgaggtatccagattctgtcgttaggactgactaggca